(19) United States
(12) Patent Application Publication
(10) Pub. No.: US 2026/0013957 A1
(43) Pub. Date: Jan. 15, 2026

(54) ENGAGEMENT OF MICROSURGICAL ROBOTIC SYSTEM

(71) Applicant: FORSIGHT ROBOTICS LTD, Yokneam Illit (IL)

(72) Inventors: Joseph NATHAN, Haifa (IL); Yoav GOLAN, Tel Aviv (IL); Oshri VAKNIN, Tel Aviv (IL); Amit DALIOT, Kokhav Ya'ir (IL)

(21) Appl. No.: 19/333,710

(22) Filed: Sep. 19, 2025

Related U.S. Application Data
(63) Continuation of application No. PCT/IB2024/052760, filed on Mar. 22, 2024.
(60) Provisional application No. 63/454,420, filed on Mar. 24, 2023.

(51) Int. Cl. A61B 34/30; A61B 34/20; A61B 90/00; A61F 9/007

(52) U.S. Cl. CPC A61B 34/30; A61B 34/20; A61F 9/007; A61B 2090/373

(57) ABSTRACT

Apparatus and methods are described for performing a procedure on a portion of a body of a patient using a surgical tool. A computer processor drives a display to show an image that includes a representation of the surgical tool. In response to a control-component tool being at least partially aligned with the representation of the surgical tool within the image upon the display, the computer processor engages the control-component tool with the surgical tool, such that the tip of the surgical tool is moved within the patient's eye in a manner that corresponds with movement of the location and orientation of the tip of the control-component tool. Other applications are also described.

### CROSS-REFERENCES TO RELATED APPLICATIONS

The present application is a continuation of PCT Application PCT/IB2024/052760 to Nathan et al., filed Mar. 22, 2024 (published as WO 24/201236), entitled “Engagement of a microsurgical robotic system,” which claims priority from U.S. Provisional Patent Application No. 63/454,420 to Nathan et al., filed Mar. 24, 2023, entitled “Engagement of a microsurgical robotic system,” which is incorporated herein by reference.

### FIELD OF EMBODIMENTS OF THE INVENTION

Some applications of the present invention generally relate to medical apparatus and methods. Specifically, some applications of the present invention relate to apparatus and methods for performing microsurgical procedures in a robotic manner.

### BACKGROUND

Cataract surgery involves the removal of the natural lens of the eye that has developed an opacification (known as a cataract), and its replacement with an intraocular lens. Such surgery typically involves a number of standard steps, which are performed sequentially.

In an initial step, the patient's face around the eye is disinfected (typically, with iodine solution), and their face is covered by a sterile drape, such that only the eye is exposed. When the disinfection and draping has been completed, the eye is anesthetized, typically using a local anesthetic, which is administered in the form of liquid eye drops. The eyeball is then exposed, using an eyelid speculum that holds the upper and lower eyelids open. One or more incisions (and typically two or three incisions) are made in the cornea of the eye. The incision(s) are typically made using a specialized blade, which is called a keratome blade. At this stage, lidocaine is typically injected into the anterior chamber of the eye, in order to further anesthetize the eye. Following this step, a viscoelastic injection is applied via the corneal incision(s). The viscoelastic injection is performed in order to stabilize the anterior chamber and to help maintain eye pressure during the remainder of the procedure, and also in order to distend the lens capsule.

In a subsequent stage, known as capsulorhexis, a part of the anterior lens capsule is removed. Various enhanced techniques have been developed for performing capsulorhexis, such as laser-assisted capsulorhexis, zepto-rhexis (which utilizes precision nano-pulse technology), and marker-assisted capsulorhexis (in which the cornea is marked using a predefined marker, in order to indicate the desired size for the capsule opening).

Subsequently, it is common for a fluid wave to be injected via the corneal incision, in order to dissect the cataract's outer cortical layer, in a step known as hydrodissection. In a subsequent step, known as hydrodelineation, the outer softer epi-nucleus of the lens is separated from the inner firmer endo-nucleus by the injection of a fluid wave. In the next step, ultrasonic emulsification of the lens is performed, in a process known as phacoemulsification. The nucleus of the lens is broken initially using a chopper, following which the outer fragments of the lens are broken and removed, typically using an ultrasonic phacoemulsification probe. Further typically, a separate tool is used to perform suction during the phacoemulsification. When the phacoemulsification is complete, the remaining lens cortex (i.e., the outer layer of the lens) material is aspirated from the capsule. During the phacoemulsification and the aspiration, aspirated fluids are typically replaced with irrigation of a balanced salt solution, in order to maintain fluid pressure in the anterior chamber. In some cases, if deemed to be necessary, then the capsule is polished. Subsequently, the intraocular lens (IOL) is inserted into the capsule. The IOL is typically foldable and is inserted in a folded configuration, before unfolding inside the capsule. At this stage, the viscoelastic is removed, typically using the suction device that was previously used to aspirate fluids from the capsule. If necessary, the incision(s) is sealed by elevating the pressure inside the bulbus oculi (i.e., the globe of the eye), causing the internal tissue to be pressed against the external tissue of the incision, such as to force closed the incision.

### SUMMARY

In accordance with some applications of the present invention, a robotic system is configured for use in a microsurgical procedure, such as intraocular surgery. Typically, when used for intraocular surgery, the robotic system includes one or more robotic units (which are configured to hold tools), in addition to an imaging system, one or more displays and a control-component unit (e.g., control-component unit that includes a pair of control components), via which one or more operators (e.g., healthcare professionals, such as a physician and/or a nurse) are able to control the robotic units. Typically, the robotic system includes one or more computer processors, via which components of the system and operator(s) operatively interact with each other.

Typically, movement of the robotic units (and/or control of other aspects of the robotic system) is at least partially controlled by the one or more operators. For example, the operator may receive images of the patient's eye and the robotic units and/or tools disposed therein, via the display. Typically, such images are acquired by the imaging system. For some applications, the imaging system is a stereoscopic imaging device and the display is a stereoscopic display. Based on the received images, the operator typically performs steps of the procedure. For some applications, the operator provides commands to the robotic units via the control-component unit. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), forceps (e.g., opening and closing of forceps), an intraocular-lens-manipulator tool (e.g., such that the tool manipulates the intraocular lens inside the eye for precise positioning of the intraocular lens within the eye), and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, orientation, and/or XYZ positioning of the imaging system).

Typically, the control-component unit includes one or more control components that are configured to correspond to respective robotic units of the robotic system. For example, the system may include first and second robotic units, and the control-component unit may include first and second control components, as shown. Typically, each of the control components is an arm that includes a plurality of links that are coupled to each other via joints. For some applications, the control-components include respective control-component tools (that are typically configured to replicate the robotic units). Typically, the computer processor determines the XYZ location and orientation of the tip of the control-component tool, and drives the robotic unit such that the tip of the actual tool that is being used to perform the procedure (i.e., the surgical tool) tracks the movements of the tip of the control-component tool and such that changes in the orientation of the surgical tool track changes in the orientation of the control-component tool. For some applications, movement of the control-component tool by the operator is scaled up or down by the computer processor, as described in further detail hereinbelow.

It is typically desirable that the control-component tool becomes engaged with the surgical tool of the robotic unit (such that the movements of the control-component tool control movement of the surgical tool) with the orientations of the surgical tool and the control-component tool (within their respective frames of reference) being substantially similar to each other. If the orientations of the surgical tool and the control-component tool (within their respective frames of reference) are dissimilar from each other, this can lead to the operator being disoriented, which may in turn lead to discomfort, extended surgical durations, and erroneous movements due to the operator's disorientation.

Typically, the control of the surgical tool by the operator has limitations. For example, the workspace in which the operator can move the control-component tool (referred to hereinafter “the control-component workspace”) is typically physically constrained by where it is comfortable or even possible for the operator to move the control-component tool. In addition, the workspace of the surgical tool (referred to hereinafter “the tool workspace”) is typically physically constrained by the space within which it is possible for the robotic arm to move the surgical tool. Typically, in cases in which there are a plurality of control components and a corresponding plurality of surgical tools, each of the control-component tools has a respective control-component workspace, and each of the surgical tools has a respective tool workspace. In some cases, one of the limitations on the control-component workspace for one of the control-component tools is that it impinges on the control-component workspace of a second control-component tool. Similarly, in some cases, one of the limitations on the tool workspace for one of the surgical tools is that it impinges on the tool workspace of a second one of the surgical tools.

The control-component workspace should be such that the control-component tool has sufficient freedom of movement such as to have the ability to control movement of the surgical tool within the surgical space. If the operator assumes control of the surgical tool (via the control-component tool) when the control-component tool is close to the edge of the control-component workspace, the movement of the control-component tool (and therefore that of the surgical tool) will be limited. Therefore, it is typically preferable for the operator to engage the control-component tool with the surgical tool when the control-component tool is positioned and oriented such that the operator has good freedom of movement of the control-component tool.

The tool workspace should ideally cover the space within which the tool is expected to be manipulated for the purpose of the surgery (hereinafter “the surgical space”). If the operator assumes control of the surgical tool (via the control-component tool) when the surgical tool is at the edge of the tool workspace, the movement of the surgical tool will be limited. Therefore, it is typically preferable for the operator to engage the control-component tool with the surgical tool when the surgical tool is positioned and oriented such that it has good freedom of movement.

In other words, the operator should be able to freely move the surgical tool to all positions and orientation within the surgical space, without the control-component tool reaching the limits of the control-component workspace and without the surgical tool reaching the limits of the tool workspace.

In accordance with some applications of the present invention, the control-component tool becomes engaged with the surgical tool (such that the movements of the control-component tool control movement of the surgical tool) with the orientation of the surgical tool and the control-component tool being substantially similar to each other (within their respective frames of reference), thereby avoiding disorientation of the operator. For some applications, the control-component tool becomes engaged with the surgical tool, when the surgical tool and the control-component tool are toward the centers of the tool workspace and the control-component workspace, respectively. Typically, the operator is able to engage the surgical tool and the control-component tool with respect to each other and/or disengage the surgical tool and the control-component tool from each other using standard movements of the control-component tool and without requiring additional external inputs.

There is therefore provided, in accordance with some applications of the present invention, apparatus for performing a procedure on a portion of a body of a patient using a surgical tool that has a tip, an imaging system and a display, the apparatus including:
a robotic unit configured to move the surgical tool;
a control-component unit that includes one or more location sensors and a control-component tool that is configured to be moved by an operator and that defines a tip; and
at least one computer processor configured:
   to drive the display to show an image of the surgical tool and the portion of the patient's body;
   in response to the control-component tool being at least partially aligned with the surgical tool within the image upon the display, to engage the control-component tool with the surgical tool, and
   when the control-component tool is engaged with the surgical tool:
      to determine movement of the location and orientation of the tip of the control-component tool based upon data received from the one or more location sensors; and
      to move the tip of the surgical tool within the patient's eye in a manner that corresponds with the movement of the location and orientation of the tip of the control-component tool.

In some applications, the apparatus is configured for performing an ophthalmic procedure on an eye of a patient using one or more ophthalmic tools that have tips and the robotic unit is configured to move the one or more ophthalmic tools within the patient's eye.

In some applications, the computer processor is configured to drive the display to display an augmented surgical tool overlaid upon the surgical tool upon the display.

In some applications, the computer processor is configured to drive the display to display an augmented control-component tool overlaid upon the control-component tool upon the display, and the computer processor is configured to engage the control-component tool with the surgical tool in response to the augmented control-component tool being at least partially aligned with the surgical tool within the image upon the display.

In some applications, the computer processor is configured to automatically move the control-component tool to become at least partially aligned with the surgical tool within the image upon the display, such that the control-component tool becomes engaged with the surgical tool.

In some applications, the robotic unit is capable of moving the surgical tool within a tool workspace, and the computer processor is configured to automatically drive the robotic unit to move the surgical tool to an initial position at which the surgical tool is within a given portion of the tool workspace.

In some applications:
the robotic unit is configured to move the surgical tool within a tool frame of reference;
the control-component tool is moveable within a control-component frame of reference; and
the computer processor is configured to guide the operator to move the control-component tool to become engaged with the surgical tool when the control-component tool is disposed at an orientation within the control component frame of reference that is substantially similar to an orientation of the surgical tool within the tool frame of reference.

In some applications, the computer processor is configured to engage the control-component tool with the surgical tool, without requiring any inputs via any operator-controlled interfaces other than movement of the control-component tool.

In some applications:
the surgical tool includes left and right surgical tools;
the robotic unit includes left and right robotic units configured to move the left and right surgical tools respectively;
the control-component unit includes left and right control-component tools that are configured to be moved by the operator and that define tips;
the left control-component tool is engageable with both of the left and right surgical tools; and
the right control-component tool is engageable with both of the left and right surgical tools.

In some applications, the computer processor is configured to engage the left control-component tool with the right surgical tool and the right control-component tool with the left surgical tool in response to an input from the operator.

In some applications, the computer processor is configured to engage the left control-component tool with the right surgical tool and the right control-component tool with the left surgical tool automatically based on positions of the left and right robotic units relative to the portion of the patient's body.

In some applications, the robotic unit is capable of moving the surgical tool within a tool workspace, and the computer processor is configured to disengage the control-component tool from the surgical tool in response to the surgical tool being moved toward an edge of the tool workspace.

In some applications, the computer processor is configured to generate a graphic on the display indicating that the control-component tool is being disengaged from the surgical tool.

In some applications, the control-component tool is moveable within a control-component workspace, and the computer processor is configured to guide the operator to move the control-component tool to become engaged with the surgical tool when the control-component tool is disposed within a given portion of the control-component workspace.

In some applications, the computer processor is configured to guide the operator to move the control-component tool to become engaged with the surgical tool when the control-component tool is disposed relatively centrally within the control-component workspace.

In some applications, the robotic unit is capable of moving the surgical tool within a tool workspace, and the computer processor is configured to guide the operator to move the control-component tool to become engaged with the surgical tool when the control-component tool is disposed such that the control-component tool can be moved so as to move the surgical tool to any location within the tool workspace without the control-component tool leaving the control-component workspace.

There is further provided, in accordance with some applications of the present invention, apparatus for performing a procedure on a portion of a body of a patient using a surgical tool that has a tip, an imaging system and a display, the apparatus including:
a robotic unit configured to move the surgical tool;
a control-component unit that includes one or more location sensors and a control-component tool that is configured to be moved by an operator, that defines a tip, and that is moveable within a control-component workspace; and
at least one computer processor configured:
   to guide the operator to move the control-component tool to become engaged with the surgical tool when the control-component tool is disposed within a given portion of the control-component workspace, and
   when the control-component tool is engaged with the surgical tool:
      to determine movement of the location and orientation of the tip of the control-component tool based upon data received from the one or more location sensors; and
      to move the tip of the surgical tool within the patient's eye in a manner that corresponds with the movement of the location and orientation of the tip of the control-component tool.

In some applications, the computer processor is configured to guide the operator to move the control-component tool to become engaged with the surgical tool when the control-component tool is disposed relatively centrally within the control-component workspace.

In some applications, the robotic unit is capable of moving the surgical tool within a tool workspace, and the computer processor is configured to guide the operator to move the control-component tool to become engaged with the surgical tool when the control-component tool is disposed such that the control-component tool can be moved such as to move the surgical tool to any location within the tool workspace without the control-component tool leaving the control-component workspace.

In some applications, the apparatus is configured for performing an ophthalmic procedure on an eye of a patient using one or more ophthalmic tools that have tips and the robotic unit is configured to move the one or more ophthalmic tools within the patient's eye.

In some applications:
the robotic unit is configured to move the surgical tool within a tool frame of reference;
the control-component tool is moveable within a control-component frame of reference; and
the computer processor is configured to guide the operator to move the control-component tool to become engaged with the surgical tool when the control-component tool is disposed at an orientation within the control component frame of reference that is substantially similar to an orientation of the surgical tool within the tool frame of reference.

In some applications, the computer processor is configured to engage the control-component tool with the surgical tool, without requiring any inputs via any operator-controlled interfaces other than movement of the control-component tool.

In some applications:
the surgical tool includes left and right surgical tools;
the robotic unit includes left and right robotic units configured to move the left and right surgical tools respectively;
the control-component unit includes left and right control-component tools that are configured to be moved by the operator and that define tips;
the left control-component tool is engageable with both of the left and right surgical tools; and
the right control-component tool is engageable with both of the left and right surgical tools.

In some applications, the computer processor is configured to engage the left control-component tool with the right surgical tool and the right control-component tool with the left surgical tool in response to an input from the operator.

In some applications, the computer processor is configured to engage the left control-component tool with the right surgical tool and the right control-component tool with the left surgical tool automatically based on positions of the left and right robotic units relative to the portion of the patient's body.

In some applications, the robotic unit is capable of moving the surgical tool within a tool workspace, and the computer processor is configured to disengage the control-component tool from the surgical tool in response to the surgical tool being moved toward an edge of the tool workspace.

In some applications, the computer processor is configured to generate a graphic on the display indicating that the control-component tool is being disengaged from the surgical tool.

In some applications, the computer processor is configured to drive the display to show an image of the surgical tool and the portion of the patient's body.

In some applications, the computer processor is configured to drive the display to display an augmented surgical tool overlaid upon the surgical tool upon the display.

In some applications, the computer processor is configured, in response to the control-component tool being at least partially aligned with the surgical tool within the image upon the display, to engage the control-component tool with the surgical tool.

In some applications, the computer processor is configured to automatically move the control-component tool to becoming at least partially aligned with the surgical tool within the image upon the display, such that the control-component tool becomes engaged with the surgical tool.

In some applications, the computer processor is configured to drive the display to display an augmented control-component tool overlaid upon the control-component tool upon the display, and the computer processor is configured to engage the control-component tool with the surgical tool in response to the augmented control-component tool being at least partially aligned with the surgical tool within the image upon the display.

There is further provided, in accordance with some applications of the present invention, apparatus for performing a procedure on a portion of a body of a patient using a surgical tool that has a tip, an imaging system and a display, the apparatus including:
a robotic unit configured to move the surgical tool within a tool frame of reference;
a control-component unit that includes one or more location sensors and a control-component tool that is configured to be moved by an operator, that defines a tip, and that is moveable within a control-component frame of reference; and
at least one computer processor configured:
   to guide the operator to move the control-component tool to become engaged with the surgical tool when the control-component tool is disposed at an orientation within the control-component frame of reference that is substantially similar to an orientation of the surgical tool within the tool frame of reference, and
   when the control-component tool is engaged with the surgical tool:
      to determine movement of the location and orientation of the tip of the control-component tool based upon data received from the one or more location sensors; and
      to move the tip of the surgical tool within the patient's eye in a manner that corresponds with the movement of the location and orientation of the tip of the control-component tool.

In some applications, the apparatus is configured for performing an ophthalmic procedure on an eye of a patient using one or more ophthalmic tools that have tips and the robotic unit is configured to move the one or more ophthalmic tools within the patient's eye.

In some applications, the computer processor is configured to engage the control-component tool with the surgical tool, without requiring any inputs via any operator-controlled interfaces other than movement of the control-component tool.

In some applications:
the surgical tool includes left and right surgical tools;
the robotic unit includes left and right robotic units configured to move the left and right surgical tools respectively;
the control-component unit includes left and right control-component tools that are configured to be moved by the operator and that define tips;
the left control-component tool is engageable with both of the left and right surgical tools; and
the right control-component tool is engageable with both of the left and right surgical tools.

In some applications, the computer processor is configured to engage the left control-component tool with the right surgical tool and the right control-component tool with the left surgical tool in response to an input from the operator.

In some applications, the computer processor is configured to engage the left control-component tool with the right surgical tool and the right control-component tool with the left surgical tool automatically based on positions of the left and right robotic units relative to the portion of the patient's body.

In some applications, the robotic unit is capable of moving the surgical tool within a tool workspace, and the computer processor is configured to disengage the control-component tool from the surgical tool in response to the surgical tool being moved toward an edge of the tool workspace.

In some applications, the computer processor is configured to generate a graphic on the display indicating that the control-component tool is being disengaged from the surgical tool.

In some applications, the computer processor is configured to drive the display to show an image of the surgical tool and the portion of the patient's body.

In some applications, the computer processor is configured to drive the display to display an augmented surgical tool overlaid upon the surgical tool upon the display.

In some applications, the computer processor is configured, in response to the control-component tool being at least partially aligned with the surgical tool within the image upon the display, to engage the control-component tool with the surgical tool.

In some applications, the computer processor is configured to automatically move the control-component tool to becoming at least partially aligned with the surgical tool within the image upon the display, such that the control-component tool becomes engaged with the surgical tool.

In some applications, the computer processor is configured to drive the display to display an augmented control-component tool overlaid upon the control-component tool upon the display, and the computer processor is configured to engage the control-component tool with the surgical tool in response to the augmented control-component tool being at least partially aligned with the surgical tool within the image upon the display.

In some applications, the control-component is moveable within a control-component workspace, and the computer processor is configured to guide the operator to move the control-component tool to become engaged with the surgical tool when the control-component tool is disposed within a given portion of the control-component workspace.

In some applications, the computer processor is configured to guide the operator to move the control-component tool to become engaged with the surgical tool when the control-component tool is disposed relatively centrally within the control-component workspace.

In some applications, the robotic unit is capable of moving the surgical tool within a tool workspace, and the computer processor is configured to guide the operator to move the control-component tool to become engaged with the surgical tool when the control-component tool is disposed such that the control-component tool can be moved such as to move the surgical tool to any location within the tool workspace without the control-component tool leaving the control-component workspace.

There is further provided, in accordance with some applications of the present invention, apparatus for performing a procedure on a portion of a body of a patient using a surgical tool that has a tip, an imaging system and a display, the apparatus including:
a robotic unit configured to move the surgical tool;
a control-component unit that includes one or more location sensors and a control-component tool that is configured to be moved by an operator and that defines a tip; and
at least one computer processor configured:
   to automatically move the control-component tool to an engagement position and orientation at which the control-component tool becomes engaged with the surgical tool, and
   when the control-component tool is engaged with the surgical tool:
      to determine movement of the location and orientation of the tip of the control-component tool based upon data received from the one or more location sensors; and
      to move the tip of the surgical tool within the patient's eye in a manner that corresponds with the movement of the location and orientation of the tip of the control-component tool.

In some applications, the apparatus is configured for performing an ophthalmic procedure on an eye of a patient using one or more ophthalmic tools that have tips and the robotic unit is configured to move the one or more ophthalmic tools within the patient's eye.

In some applications, the robotic unit is capable of moving the surgical tool within a tool workspace, and the computer processor is configured to automatically drive the robotic unit to move the surgical tool to an initial position at which the surgical tool is within a given portion of the tool workspace.

In some applications, the computer processor is configured to engage the control-component tool with the surgical tool, without requiring any inputs via any operator-controlled interfaces other than movement of the control-component tool.

In some applications:
the robotic unit is configured to move the surgical tool within a tool frame of reference;
the control-component tool is moveable within a control-component frame of reference; and
the computer processor is configured to guide the operator to move the control-component tool to become engaged with the surgical tool when the control-component tool is disposed at an orientation within the control component frame of reference that is substantially similar to an orientation of the surgical tool within the tool frame of reference.

In some applications:
the surgical tool includes left and right surgical tools;
the robotic unit includes left and right robotic units configured to move the left and right surgical tools respectively;
the control-component unit includes left and right control-component tools that are configured to be moved by the operator and that define tips;
the left control-component tool is engageable with both of the left and right surgical tools; and
the right control-component tool is engageable with both of the left and right surgical tools.

In some applications, the computer processor is configured to engage the left control-component tool with the right surgical tool and the right control-component tool with the left surgical tool in response to an input from the operator.

In some applications, the computer processor is configured to engage the left control-component tool with the right surgical tool and the right control-component tool with the left surgical tool automatically based on positions of the left and right robotic units relative to the portion of the patient's body.

In some applications, the robotic unit is capable of moving the surgical tool within a tool workspace, and the computer processor is configured to disengage the control-component tool from the surgical tool in response to the surgical tool being moved toward an edge of the tool workspace.

In some applications, the computer processor is configured to generate a graphic on the display indicating that the control-component tool is being disengaged from the surgical tool.

In some applications, the computer processor is configured to drive the display to show an image of the surgical tool and the portion of the patient's body.

In some applications, the computer processor is configured to drive the display to display an augmented surgical tool overlaid upon the surgical tool upon the display.

In some applications, the computer processor is configured, in response to the control-component tool being at least partially aligned with the surgical tool within the image upon the display, to engage the control-component tool with the surgical tool.

In some applications, the computer processor is configured to drive the display to display an augmented control-component tool overlaid upon the control-component tool upon the display, and the computer processor is configured to engage the control-component tool with the surgical tool in response to the augmented control-component tool being at least partially aligned with the surgical tool within the image upon the display.

In some applications, the control-component tool is moveable within a control-component workspace, and the computer processor is configured to guide the operator to move the control-component tool to become engaged with the surgical tool when the control-component tool is disposed within a given portion of the control-component workspace.

In some applications, the computer processor is configured to guide the operator to move the control-component tool to become engaged with the surgical tool when the control-component tool is disposed relatively centrally within the control-component workspace.

In some applications, the robotic unit is capable of moving the surgical tool within a tool workspace, and the computer processor is configured to guide the operator to move the control-component tool to become engaged with the surgical tool when the control-component tool is disposed such that the control-component tool can be moved such as to move the surgical tool to any location within the tool workspace without the control-component tool leaving the control-component workspace.

There is further provided, in accordance with some applications of the present invention, apparatus for performing a procedure on a portion of a body of a patient using a surgical tool that has a tip, an imaging system and a display, the apparatus including:
a robotic unit configured to move the surgical tool;
a control-component unit that includes one or more location sensors and a control-component tool that is configured to be moved by an operator and that defines a tip; and
a computer processor configured:
   to receive an input that control-component tool should become engaged with the surgical tool, the input including movement of the control-component tool to a given position and orientation, and
   when the control-component tool is engaged with the surgical tool:
      to determine movement of the location and orientation of the tip of the control-component tool based upon data received from the one or more location sensors; and
      to move the tip of the surgical tool within the patient's eye in a manner that corresponds with the movement of the location and orientation of the tip of the control-component tool.

In some applications, the computer processor is configured to receive the input that control-component tool should become engaged with the surgical tool, without requiring any inputs via any operator-controlled interfaces other than movement of the control-component tool.

In some applications, the apparatus is configured for performing an ophthalmic procedure on an eye of a patient using one or more ophthalmic tools that have tips and the robotic unit is configured to move the one or more ophthalmic tools within the patient's eye.

In some applications, the robotic unit is capable of moving the surgical tool within a tool workspace, and the computer processor is configured to automatically drive the robotic unit to move the surgical tool to an initial position at which the surgical tool is within a given portion of the tool workspace.

In some applications:
the robotic unit is configured to move the surgical tool within a tool frame of reference;
the control-component tool is moveable within a control-component frame of reference; and
the computer processor is configured to guide the operator to move the control-component tool to become engaged with the surgical tool when the control-component tool is disposed at an orientation within the control component frame of reference that is substantially similar to an orientation of the surgical tool within the tool frame of reference.

In some applications:
the surgical tool includes left and right surgical tools;
the robotic unit includes left and right robotic units configured to move the left and right surgical tools respectively;
the control-component unit includes left and right control-component tools that are configured to be moved by the operator and that define tips;
the left control-component tool is engageable with both of the left and right surgical tools; and
the right control-component tool is engageable with both of the left and right surgical tools.

In some applications, the computer processor is configured to engage the left control-component tool with the right surgical tool and the right control-component tool with the left surgical tool in response to an input from the operator.

In some applications, the computer processor is configured to engage the left control-component tool with the right surgical tool and the right control-component tool with the left surgical tool automatically based on positions of the left and right robotic units relative to the portion of the patient's body.

In some applications, the robotic unit is capable of moving the surgical tool within a tool workspace, and the computer processor is configured to disengage the control-component tool from the surgical tool in response to the surgical tool being moved toward an edge of the tool workspace.

In some applications, the computer processor is configured to generate a graphic on the display indicating that the control-component tool is being disengaged from the surgical tool.

In some applications, the computer processor is configured to drive the display to show an image of the surgical tool and the portion of the patient's body.

In some applications, the computer processor is configured to drive the display to display an augmented surgical tool overlaid upon the surgical tool upon the display.

In some applications, the computer processor is configured, in response to the control-component tool being at least partially aligned with the surgical tool within the image upon the display, to engage the control-component tool with the surgical tool.

In some applications, the computer processor is configured to drive the display to display an augmented control-component tool overlaid upon the control-component tool upon the display, and the computer processor is configured to engage the control-component tool with the surgical tool in response to the augmented control-component tool being at least partially aligned with the surgical tool within the image upon the display.

In some applications, the control-component tool is moveable within a control-component workspace, and the computer processor is configured to guide the operator to move the control-component tool to become engaged with the surgical tool when the control-component tool is disposed within a given portion of the control-component workspace.

In some applications, the computer processor is configured to guide the operator to move the control-component tool to become engaged with the surgical tool when the control-component tool is disposed relatively centrally within the control-component workspace.

In some applications, the robotic unit is capable of moving the surgical tool within a tool workspace, and the computer processor is configured to guide the operator to move the control-component tool to become engaged with the surgical tool when the control-component tool is disposed such that the control-component tool can be moved such as to move the surgical tool to any location within the tool workspace without the control-component tool leaving the control-component workspace.

There is further provided, in accordance with some applications of the present invention, apparatus for performing a procedure on a portion of a body of a patient using a surgical tool that has a tip, an imaging system and a display, the apparatus including:
a robotic unit configured to move the surgical tool within a tool workspace;
a control-component unit that includes:
   a control-component tool that is configured to be moved by an operator and that defines a tip; and
   an inertial measurement unit including at least one of sensor selected from the group consisting of: a three-axis accelerometer, a three-axis gyroscope, and a three-axis magnetometer, the inertial measurement unit being configured to generate inertial-measurement-unit data indicative of an orientation of the tip of control-component tool; a computer processor configured:
      determine the location and the orientation of the tip of the control-component tool based upon data received from the one or more location sensors;
      move the tip of the ophthalmic tool within the patient's eye in a manner that corresponds with movement of the control-component tool; and
      recalibrate the inertial measurement unit in response to the control-component tool being docked at a known orientation within the control-component unit.

In some applications:
the control-component unit includes a plurality of links that are coupled to each other via a plurality of rotational axes; and
the control-component tool is coupled to the links and is configured to be moved by an operator such that as the operator moves the control-component tool along linear X, Y, and Z directions, the links rotate around the rotational axes, and
   the plurality of links include an X-direction link through which X-direction linear motion is effected and a Z rotational axis about which movement in the Z-direction is effected, and
   the X-direction link is aligned with the Z rotational axis, such that the X-direction link does not exert any torque about the Z rotational axis.

In some applications:
the control-component unit includes X, Y and Z linear-motion rotational axes, and pitch, roll and yaw angular-motion rotational axes,
the control-component tool is coupled to the X, Y and Z linear-motion rotational axes and the pitch, roll and yaw angular-motion rotational axes and is configured to be moved by an operator such that:
   as the operator moves the control-component tool along linear X, Y, and Z directions, rotational motion is generated about X, Y, and Z linear-motion rotational axes, and
   as the operator moves the control-component tool through roll, pitch and yaw angular motions, rotational motion is generated about respective pitch, roll and yaw angular-motion rotational axes;
the control-component unit includes a plurality of direct-drive motors that are operatively coupled to respective X, Y, and Z linear-motion rotational axes; and
the computer processor is configured to provide force feedback to the operator via the control-component tool using the plurality of direct-drive motors.

In some applications:
the control-component unit includes X, Y and Z linear-motion rotational axes, and pitch, roll and yaw angular-motion rotational axes,
the control-component tool is coupled to the X, Y and Z linear-motion rotational axes and the pitch, roll and yaw angular-motion rotational axes and is configured to be moved by an operator such that:
   as the operator moves the control-component tool along linear X, Y, and Z directions, rotational motion is generated about X, Y, and Z linear-motion rotational axes, and
   as the operator moves the control-component tool through roll, pitch and yaw angular motions, rotational motion is generated about respective pitch, roll and yaw angular-motion rotational axes;
the control-component unit includes X-direction, Y-direction, and Z-direction motors that are operatively coupled respectively to X, Y, and Z linear-motion rotational axes and a first end of the Y-direction motor is aligned with the X rotational axis; and
the computer processor is configured to provide force feedback to the operator via the control-component tool using the X-direction, Y-direction, and Z-direction motors.

In some applications:
the control-component unit includes X, Y and Z linear-motion rotational axes, and pitch, roll and yaw angular-motion rotational axes,
the control-component tool is coupled to the X, Y and Z linear-motion rotational axes and the pitch, roll and yaw angular-motion rotational axes and is configured to be moved by an operator such that:
   as the operator moves the control-component tool along linear X, Y, and Z directions, rotational motion is generated about X, Y, and Z linear-motion rotational axes, and
   as the operator moves the control-component tool through roll, pitch and yaw angular motions, rotational motion is generated about respective pitch, roll and yaw angular-motion rotational axes, and
the control-component tool is substantially balanced around the X, Y and Z linear-motion rotational axes, and pitch, roll and yaw angular-motion rotational axes.

In some applications:
within four degrees of freedom, the control-component tool is self-balancing, and
within two degrees of freedom, the control-component unit includes counterweights such as to balance weight of the control-component and/or other components of the control-component unit about corresponding rotational axes.

In some applications, the control-component tool is self-balancing about the roll and yaw angular-motion rotational axes and about two of the X, Y, and Z linear-motion rotational axes, and the control-component includes first and second counterweights such as to balance weight of the control-component tool and/or other components of the control-component unit about the pitch angular-motion rotational axis and about one of the linear-motion rotational axes, respectively.

The present invention will be more fully understood from the following detailed description of embodiments thereof, taken together with the drawings, in which:

### BRIEF DESCRIPTION OF THE DRAWINGS

FIGS. 1A and 1B are schematic illustrations of a robotic system that is configured for use in a microsurgical procedure, such as intraocular surgery, in accordance with some applications of the present invention;

FIG. 2A is a schematic illustration of a display showing a surgical tool placed laterally with respect to a patient's cornea, in accordance with some applications of the present invention;

FIG. 2B is a schematic illustration of a display showing an augmented surgical tool overlaid on the laterally-placed surgical tool, such that the tip of the augmented surgical tool is placed in a vicinity of the tip of the surgical tool, in accordance with some applications of the present invention;

FIGS. 2C and 2D are schematic illustrations of a display showing an augmented control-component tool being positioned such as to overlay the augmented surgical tool in order to engage a first control-component tool with the robotic system, in accordance with some applications of the present invention;

FIG. 2E is a schematic illustration of a display showing a surgical tool placed superiorly with respect to a patient's cornea, in accordance with some applications of the present invention;

FIG. 2F is a schematic illustration of a display showing an augmented surgical tool overlaid on the superiorly-placed surgical tool, such that the tip of the augmented surgical tool is placed in a vicinity of the tip of the surgical tool, in accordance with some applications of the present invention;

FIGS. 2G and 2H are schematic illustrations of a display showing an augmented control-component tool being positioned such as to overlay the augmented surgical tool in order to engage a second control-component tool with the robotic system, in accordance with some applications of the present invention;

FIG. 2I is a schematic illustration of a display showing an augmented control-component tool being moved away from an augmented surgical tool in order to disengage a control-component tool from the robotic system, in accordance with some applications of the present invention;

FIG. 3A is a schematic illustration of the robotic system marked with cuboids that indicate workspaces of a control component and a surgical tool for illustrative purposes, in accordance with some applications of the present invention;

FIG. 3B is a schematic illustration of the robotic system annotated with several frames of references for illustrative purposes, in accordance with some applications of the present invention;

FIGS. 4A, 4B, 4C, and 4D are schematic illustrations of a control component and a control-component tool of a control-component unit, in accordance with some applications of the present invention; and

FIGS. 5A, 5B, 5C, and 5D are schematic illustrations of a control component and a control-component tool of a control-component unit, in accordance with some alternative applications of the present invention.

### DETAILED DESCRIPTION OF EMBODIMENTS

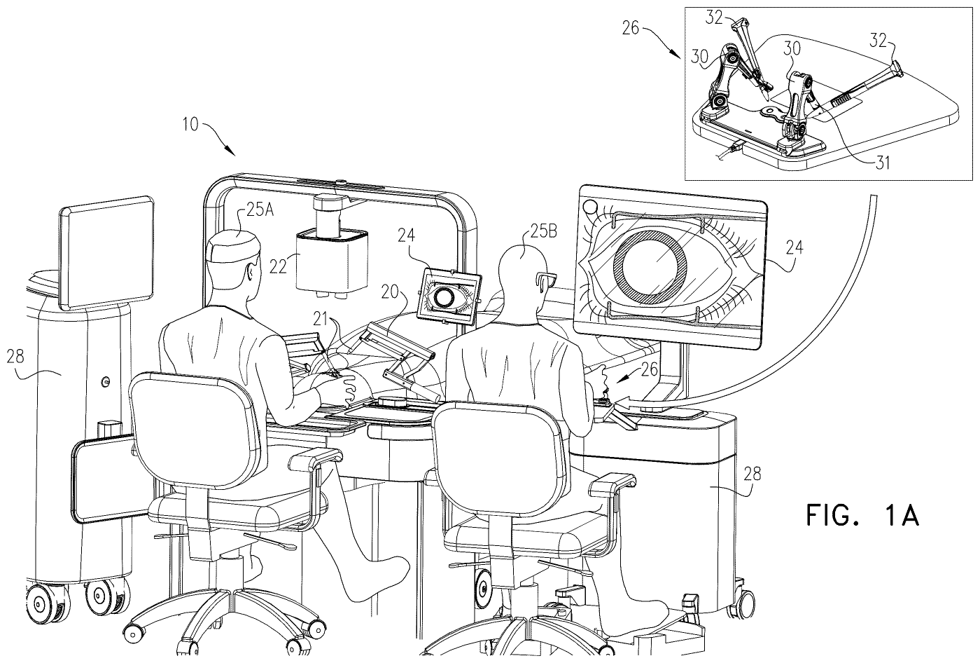

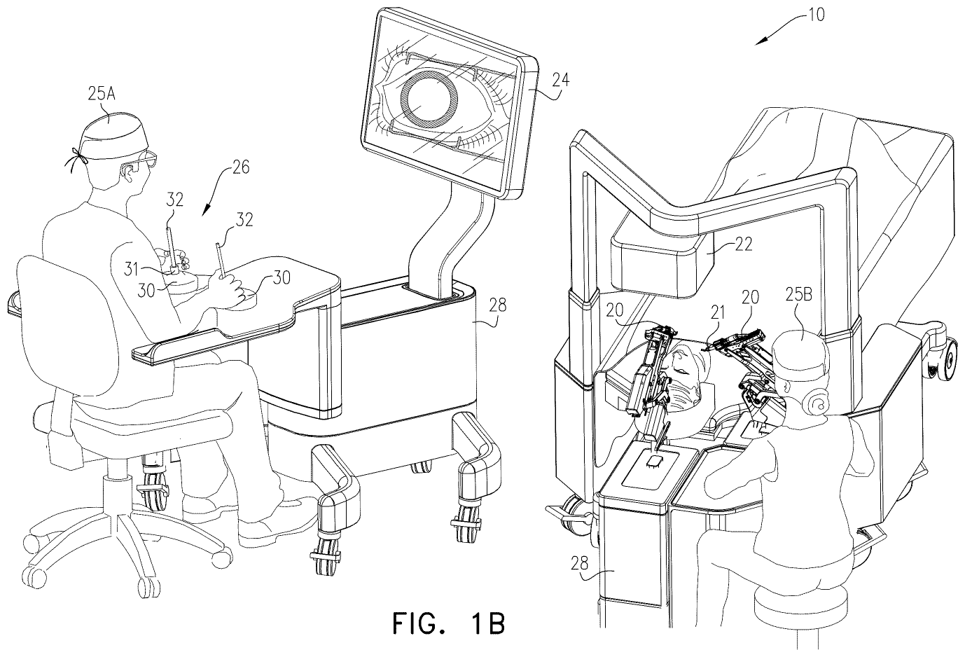

Reference is now made to FIGS. 1A and 1B, which are schematic illustrations of a robotic systems 10 that is configured for use in a microsurgical procedure, such as intraocular surgery, in accordance with some applications of the present invention. Typically, when used for intraocular surgery, robotic system 10 includes one or more robotic units 20 (which are configured to hold tools 21), in addition to an imaging system 22, one or more displays 24 and a control-component unit 26 (e.g., control-component unit that includes a pair of control components, as shown in the enlarged portion of FIG. 1A), via which one or more operators (e.g., healthcare professionals, such as a physician 25A and/or a nurse 25B) are able to control robotic units 20. Typically, robotic system 10 includes one or more computer processors 28, via which components of the system and operator(s) 25 operatively interact with each other.

FIGS. 1A and 1B show different setups of a robotic system 10 that is configured for ophthalmic surgery. As shown, in the configuration shown in FIG. 1A first and second robotic units are disposed at respective lateral positions (i.e., left and right) with respect to the eye that is being operated on, such that tools 21 that are held by the robotic units are disposed at approximately 180 degrees from each other. The configuration shown in FIG. 1B shows a first robotic unit that is placed laterally with respect to the eye and a second robotic unit positioned in a superior position with respect to the eye, such that tools 21 that are held by the robotic units are disposed at approximately 90 degrees from each other. (In the context of ophthalmic procedures, the lateral position shown in FIG. 1B is referred to as the “temporal” position. As such, the terms “lateral” and “temporal” are used interchangeably in the present application.) In some cases (not shown), the first robotic unit is placed laterally with respect to the eye and the second robotic unit positioned in an inferior position with respect to the eye, such that tools 21 that are held by the robotic units are disposed at approximately 90 degrees from each other. In general, the scope of the present disclosure includes using any number of robotic units placed at any number of respective positions in relation to the patient, and the configurations shown in FIGS. 1A and 1B should not be interpreted as limiting the scope of the disclosure in any way.

Typically, movement of the robotic units (and/or control of other aspects of the robotic system) is at least partially controlled by one or more operators (e.g., healthcare professionals, such as a physician 25A and/or a nurse 25B). For example, the operator may receive images of the patient's eye and the robotic units and/or tools disposed therein, via display 24. Typically, such images are acquired by imaging system 22. For some applications, imaging system 22 is a stereoscopic imaging device and display 24 is a stereoscopic display. Based on the received images, the operator typically performs steps of the procedure. For some applications, the operator provides commands to the robotic units via control-component unit 26. For example, FIGS. 1A and 1B show physician 25A providing commands to the robotic units via control-component unit 26, while viewing images of the patient's eye and tools 21 upon display 24. Typically, such commands include commands that control the position and/or orientation of tools that are disposed within the robotic units, and/or commands that control actions that are performed by the tools. For example, the commands may control a blade, a phacoemulsification tool (e.g., the operation mode and/or suction power of the phacoemulsification tool), forceps (e.g., opening and closing of forceps), an intraocular-lens-manipulator tool (e.g., such that the tool manipulates the intraocular lens inside the eye for precise positioning of the intraocular lens within the eye), and/or injector tools (e.g., which fluid (e.g., viscoelastic fluid, saline, etc.) should be injected, and/or at what flow rate). Alternatively or additionally, the operator may input commands that control the imaging system (e.g., the zoom, focus, orientation, and/or XYZ positioning of the imaging system).

Typically, the control-component unit includes one or more control components 30 that are configured to correspond to respective robotic units 20 of the robotic system. For example, as shown, the system may include first and second robotic units, and the control-component unit may include first and second control components, as shown. Typically, each of the control components is an arm 31 that includes a plurality of links that are coupled to each other via joints. For some applications, the control-components include respective control-component tools 32 (that are typically configured to replicate the robotic units), as shown in FIG. 1A. Typically, the computer processor determines the XYZ location and orientation of the tip of the control-component tool 32, and drives the robotic unit such that the tip of the actual tool 21 that is being used to perform the procedure tracks the movements of the tip of the control-component tool and such that changes in the orientation of tool 21 track changes in the orientation of the control-component tool. For some applications, movement of the control-component tool by the operator is scaled up or down by the computer processor, as described in further detail hereinbelow.

In some cases, tool 21 is described herein, in the specification and in the claims, as a “surgical tool.” This term is used in order to distinguish tool 21 from control-component tool 32, and should not be interpreted as limiting the type of tool that may be used as tool 21 in any way. The term “surgical tool” should be interpreted to include any one the tools described herein and or any other types of tools that may occur to a person of ordinary skill in the art upon reading the present disclosure. Typically, for ophthalmic procedures, the surgical tool is an ophthalmic tool, e.g., one of the ophthalmic tools described hereinabove.

Typically, the right control component controls movement of the surgical tool that is toward the right of the patient's head when viewing the patient from a superior position (and which would normally be controlled by the physician's right hand), and the left control component controls movement of the surgical tool that is toward the left of the patient's head when viewing the patient from a superior position (and which would normally be controlled by the physician's left hand).

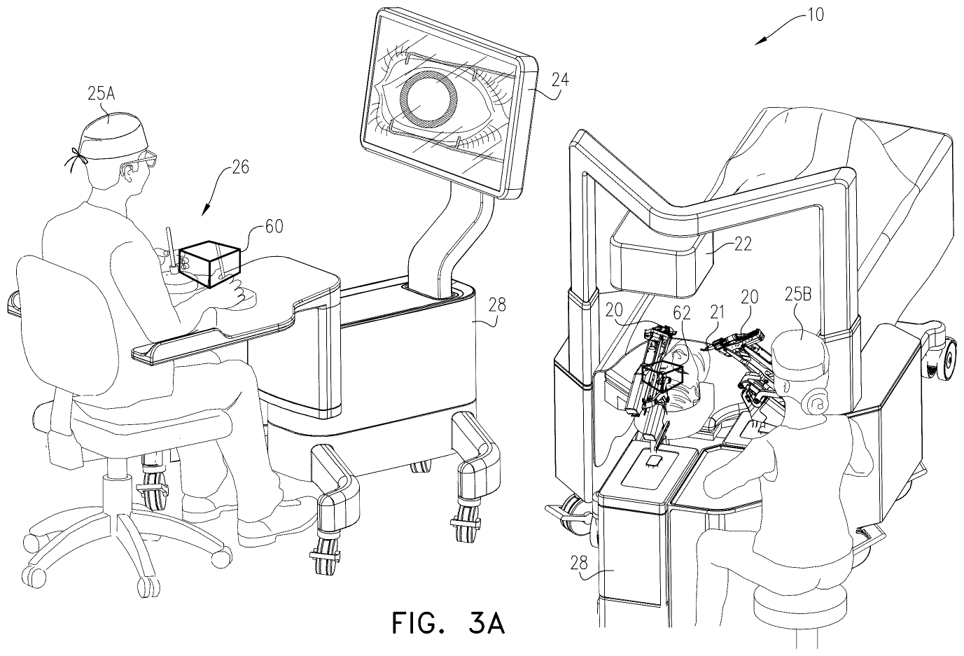

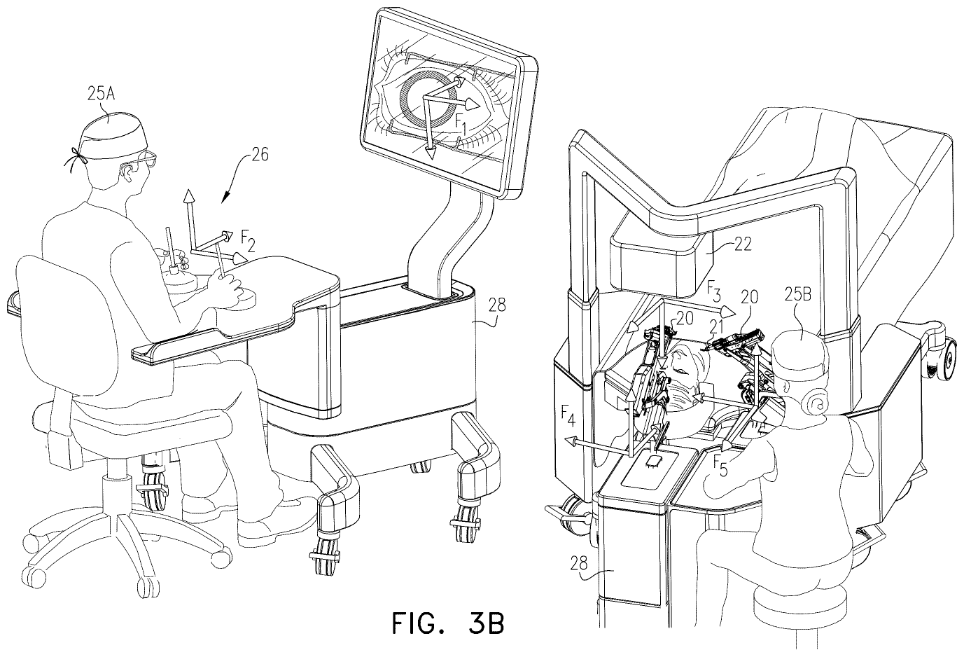

As noted above, typically, the computer processor determines the XYZ location and orientation of the tip of the control-component tool 32, and drives the robotic unit such that the tip of the actual tool 21 that is being used to perform the procedure tracks the movements of the tip of the control-component tool and such that changes in the orientation of tool 21 track changes in the orientation of the control-component tool. Thus, if the orientation of the control-component tool changes, the computer processor typically changes the orientation of the surgical tool to correspond with the change in the orientation of the control-component tool. For this purpose, it is typically desirable that the control-component tool becomes engaged with the surgical tool of the robotic unit (such that the movements of the control-component tool control movement of the surgical tool) with the orientations of the surgical tool and the control-component tool being substantially similar to each other (within their respective frames of reference (as described hereinbelow with reference to FIG. 3B)). If the orientations of the surgical tool and the control-component tool are dissimilar from each other (within their respective frames of reference (as described hereinbelow with reference to FIG. 3B)), this can lead to the operator being disoriented, which may in turn lead to discomfort, extended surgical durations, and erroneous movements due to the operator's disorientation.

The operator (e.g., physician 25A) assumes and relinquishes control of the surgical tool (via the control-component tool) multiple times during a procedure, especially when the procedure requires the use of multiple tools that are changed during surgery (as is typically the case with surgical procedures, as described above). Typically, over the course of a procedure (a) the operator assumes control of the surgical tool, (b) the operator performs surgical actions with the surgical tool, (c) the operator relinquishes control of the surgical tool, (d) the robotic system or an operator (e.g., a nurse) removes the surgical tool from the robotic unit, (e), the robotic system or the operator places a new surgical tool on the robotic unit, and steps (a)-(e) are repeated.

Typically, the control of the surgical tool by the operator has limitations. For example, the workspace in which the operator can move the control-component tool (referred to hereinafter “the control-component workspace”) is typically physically constrained by where it is comfortable or even possible for the operator to move the control-component tool. In addition, the workspace of the surgical tool (referred to hereinafter “the tool workspace”) is typically physically constrained by the space within which it is possible for the robotic arm to move the surgical tool. Typically, in cases in which there are a plurality of control components and a corresponding plurality of surgical tools, each of the control-component tools has a respective control-component workspace, and each of the surgical tools has a respective tool workspace. In some cases, one of the limitations on the control-component workspace for one of the control-component tools is that it impinges on the control-component workspace of a second control-component tool. Similarly, some cases, one of the limitations on the tool workspace for one of the surgical tools is that it impinges on the tool workspace of a second one of the surgical tools.

The control-component workspace should be such that the control-component tool has sufficient freedom of movement such as to have the ability to control movement of the surgical tool within the surgical space. If the operator assumes control of the surgical tool (via the control-component tool) when the control-component tool is close to the edge of the control-component workspace, the movement of the control-component tool (and therefore that of the surgical tool) will be limited. Therefore, it is typically preferable for the operator to engage the control-component tool with the surgical tool when the control-component tool is positioned and oriented such that the operator has good freedom of movement of the control-component tool.

The tool workspace should ideally cover the space within which the tool is expected to be manipulated for the purpose of the surgery (hereinafter “the surgical space”). If the operator assumes control of the surgical tool (via the control-component tool) when the surgical tool is at the edge of the tool workspace, the movement of the surgical tool will be limited. Therefore, it is typically preferable for the operator to engage the control-component tool with the surgical tool when the surgical tool is positioned and oriented such that it has good freedom of movement.

In other words, the operator should be able to freely move the surgical tool to all positions and orientation within the surgical space, without the control-component tool reaching the limits of the control-component workspace and without the surgical tool reaching the limits of the tool workspace.

In accordance with some applications of the present invention, the control-component tool becomes engaged with the surgical tool (such that the movements of the control-component tool control movement of the surgical tool) with the orientation of the surgical tool and the control-component tool being substantially similar to each other (within their respective frames of reference), thereby avoiding disorientation of the operator. For some applications, the control-component tool becomes engaged with the surgical tool, when the surgical tool and the control-component tool are toward the centers of the control-component workspace and the tool workspace, respectively. Typically, the operator is able to engage the surgical tool and the control-component tool to each other and/or disengage the surgical tool and the control-component tool from each other using standard movements of the control-component tool and without requiring additional external inputs.

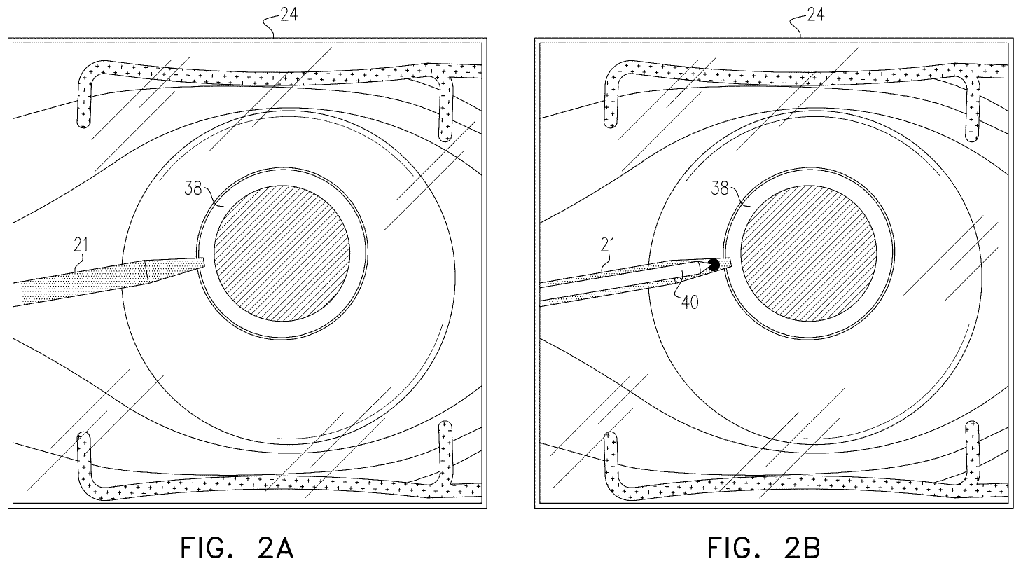

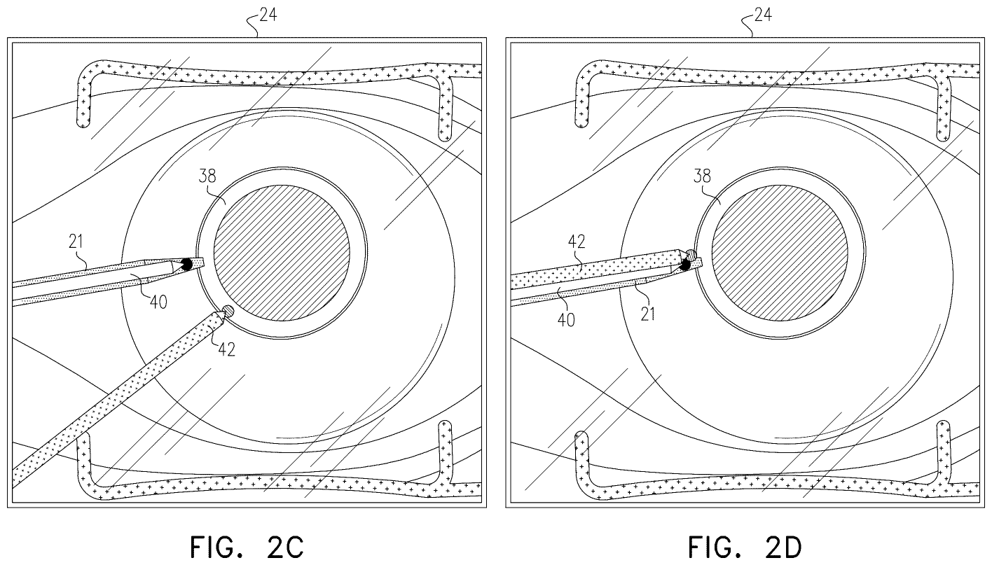

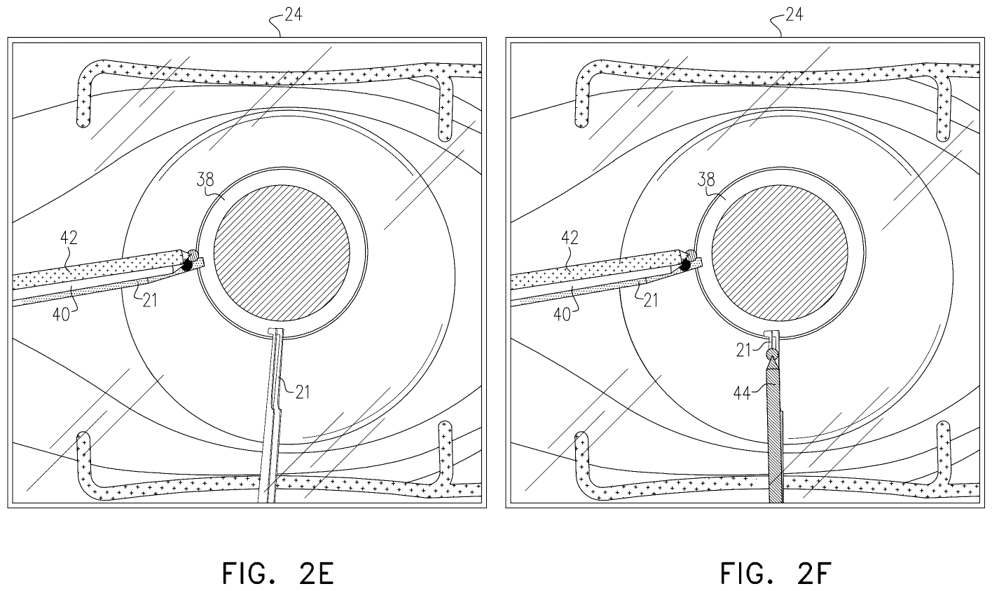

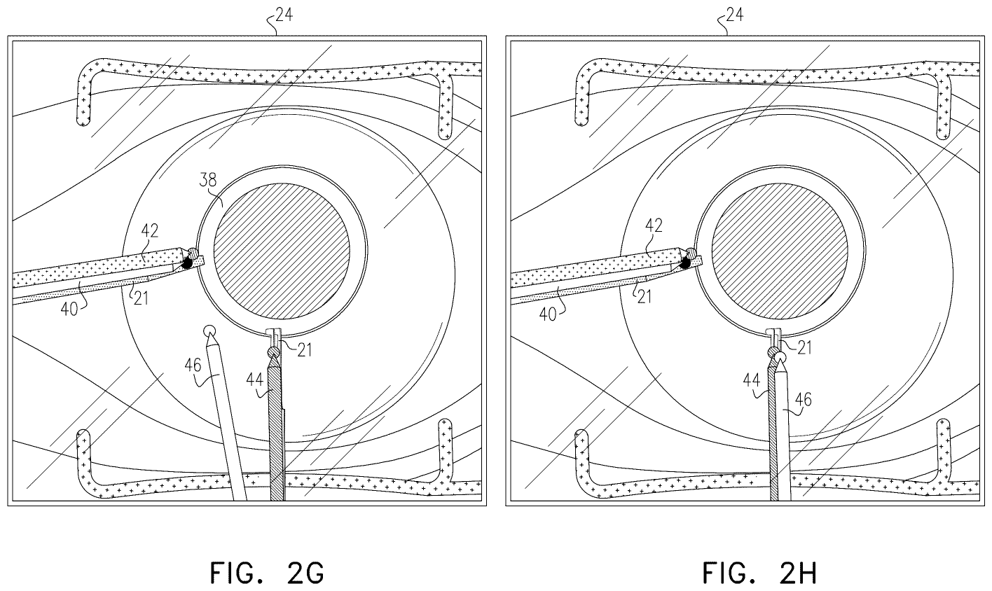

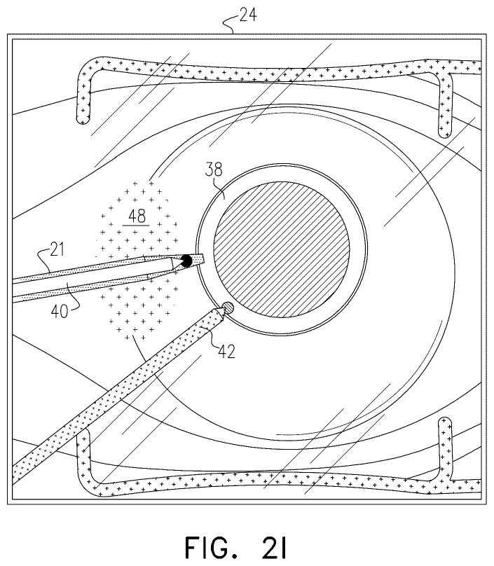

Some steps of the engagement and disengagement of the surgical tool and the control-component tool in accordance with some applications of the present invention are now described with reference to FIGS. 2A-2I.

Reference is first made to FIG. 2A is a schematic illustration of display 24 showing surgical tool 21 placed laterally with respect to a patient's cornea 38, in accordance with some applications of the present invention. For some applications, the robotic system is configured to automatically place the surgical tool in position in the vicinity of the patient's cornea (e.g., based upon images that are acquired by imaging system 22). Alternatively or additionally, the operator (e.g., nurse 25B) places the surgical tool in position in the vicinity of the patient's cornea. Typically, the surgical tool is positioned in the vicinity of the patient's cornea (by the robotic system and/or by the nurse) at a relatively central position within the tool workspace, and/or such that from this initial position within the tool workspace there is no movement of the control-component tool within the control-component workspace that would result in the tool being moved out of the tool workspace. Thus, starting from this position typically allows completion of a step of the procedure that is to be performed by the surgical tool, without the need to reposition the robotic unit during the step of the procedure.

Typically, physician 25A views the image of the robotic tool and the patient's cornea 38 on display 24. As indicated in FIGS. 1A and 1B, typically the imaging system acquires anterior images of the patient's eye, since the imaging system is typically disposed above the patient's eye. As described hereinabove, for some applications, display 24 is a three-dimensional stereoscopic display. Typically, the imaging system is configured to acquire images that encompass the entire surgical space and display 24 displays the acquired images, such that any manipulation of the surgical tools occurs within the field of view that is displayed on display 24.

Referring now to FIG. 2B, for some applications, once surgical tool 21 is positioned in the vicinity of the patient's cornea, the computer processor identifies the surgical tool and generates an augmented surgical tool 40 (i.e., an augmented image of a surgical tool) that overlays the image of the surgical tool itself. For some applications, augmented surgical tool 40 facilitates identification of the surgical tool by the physician. For some applications, an augmented surgical tool is not displayed. For some applications, the physician provides an input to the computer processor indicating whether or not she/he would like an augmented surgical tool to be displayed, and the computer processor controls the image that is displayed to the physician based on the input.

Reference is now made to FIGS. 2C and 2D, which are schematic illustrations of display 24 showing an augmented control-component tool 42 being positioned such as to overlay augmented surgical tool 40 in order to engage a first control-component tool with the robotic system, in accordance with some applications of the present invention. For some applications, when the surgical tool is placed in the vicinity of the patient's cornea, the computer processor is configured to drive display 24 to display augmented control-component tool 42, the augmented control-component tool representing the control-component tool. This augmented control-component tool is typically a virtual representation of the control-component tool.

As described hereinabove, typically the imaging system acquires anterior images of the patient's eye, since the imaging system is typically disposed above the patient's eye. Typically, the view of the patient's eye that is displayed by the display is as if the eye is being viewed from a superior position relative to the patient's head, since this is the view that a physician is accustomed to seeing during ophthalmic surgery. Display 24 typically faces the physician's face and the orientation and position of the augmented control-component tool on display 24 are rotated in accordance with the view of the eye that is shown by the display. Typically, the orientation of the augmented control-component tool within the frame of reference of the display is substantially similar to the orientation of the control-component tool within the control-component workspace frame of reference. However (due to the frame of reference of the display being different from that of the control-component workspace), the absolute position of the control-component tool is typically unrelated to the absolute position of the augmented control-component tool.

Typically, movements of the control-component tool by the physician generate corresponding movements of the augmented control-component tool on display 24. Typically, rotation of the control-component tool through angular rotations (roll, pitch, and/or yaw) generates corresponding rotations of the augmented control-component tool on display 24. Further typically, when the physician moves the control-component tool translationally (along the X, Y, or Z directions) this generates corresponding translational motion of the augmented control-component tool on display 24. For some applications, the translational motion of the augmented control-component tool on display 24 is scaled up or down relative to the translational motion of the control-component tool.

Typically, in order to engage the control-component tool with the surgical tool (such that the movements of the control-component tool control movement of the surgical tool), the physician moves the control-component tool such that the augmented control-component tool is aligned with the image of surgical tool 21 and/or augmented surgical tool 40. Typically, the augmented control-component tool is aligned with the image of surgical tool 21 and/or augmented surgical tool 40, such that (a) the tip of the augmented control-component tool overlays the image of surgical tool 21 and/or augmented surgical tool 40, and (b) the orientation of the augmented control-component tool is substantially similar to that of the image of surgical tool 21 and/or augmented surgical tool 40. Further typically, the computer processor is configured to position the augmented control-component tool such that when the surgeon aligns the augmented control-component tool with the image of surgical tool 21 and/or augmented surgical tool 40, the control-component tool itself is disposed relatively centrally within the control-component workspace, and/or such that from this initial position within the control-component workspace the control-component tool can be moved such as to move the surgical tool to any location within the tool workspace, without the control-component tool leaving the control-component workspace.

It is noted that in some applications, the above steps are performed without an augmented control-component tool being displayed. Typically, for such applications, the steps above that are described with reference to the augmented control-component tool are instead performed with an image of the control component tool itself upon the display.

It is noted that it is typically not necessary for the augmented control-component tool to be perfectly aligned with the image of surgical tool 21 and/or augmented surgical tool 40. Rather, in some applications, when the alignment is sufficiently close, any slight misalignment of position and/or orientation is maintained, such that the surgical tool does not move at the moment of engagement, and thereafter follows the surgeon's movements with slight (and typically imperceptible) misalignment. For some applications, the computer processor drives the robotic unit to adjust the position of the surgical tool such as to complete the alignment of the augmented control-component tool is aligned with the image of surgical tool 21 and/or augmented surgical tool 40.

Typically, once the augmented control-component tool is aligned with the image of surgical tool 21 and/or augmented surgical tool 40, the augmented surgical tool and/or the augmented control-component tool 40 is removed from the image that is displayed by display 24. Typically, at this stage, the control-component tool is engaged with the surgical tool (such that the movements of the control-component tool control movement of the surgical tool). Typically, movements of the control-component tool by the physician generate corresponding movements of the surgical tool. Typically, rotation of the control-component tool through angular rotations (roll, pitch, and/or yaw) generates corresponding rotations of the surgical tool. Further typically, when the physician moves the control-component tool translationally (along the X, Y, or Z directions) this generates corresponding translational motion of the surgical tool. For some applications, the translational motion of the surgical tool is scaled up or down relative to the translational motion of the control-component tool.

Reference is now made to FIGS. 2E-2H, which are schematic illustrations of generally similar steps to those described with reference to FIGS. 2A-2D being performed with respect to a second surgical tool 21 that is placed superiorly with respect to a patient's cornea 38, in accordance with some applications of the present invention. As noted above, typically, the view of the patient's eye that is displayed by the display is as if the eye is being viewed from a superior position relative to the patient's head, since this is the view that a physician is accustomed to seeing during ophthalmic surgery. Display 24 typically faces the physician's face and the orientation and position of the augmented control-component tool on display 24 are rotated in accordance with the view of the eye that is shown by the display. Thus, the second surgical tool 21 that is placed superiorly with respect to a patient's cornea 38 appears at the bottom of the display in the view shown in FIG. 2E.

For some applications, the robotic system is configured to automatically place the second surgical tool in position in the vicinity of the patient's cornea (e.g., based upon images that are acquired by imaging system 22). Alternatively or additionally, the operator (e.g., nurse 25B) places the second surgical tool in position in the vicinity of the patient's cornea. Typically, the surgical tool is positioned in the vicinity of the patient's cornea (by the robotic system and/or by the nurse) at a relatively central position within the tool workspace, and/or such that from this initial position within the tool workspace there is no movement of the control-component tool within the control-component workspace that would result in the tool being moved out of the tool workspace. Thus, starting from this position typically allows completion of a step of the procedure that is to be performed by the second surgical tool, without the need to reposition the second robotic unit during the step of the procedure.

For some applications, an augmented second surgical tool 44 is overlaid upon the image of the second surgical tool in order to facilitate identification of the surgical tool by the physician, as shown in FIG. 2F. For some applications, an augmented second surgical tool is not displayed. For some applications, the physician provides an input to the computer processor indicating whether or not she/he would like an augmented second surgical tool to be displayed, and the computer processor controls the image that is displayed to the physician based on the input.

For some applications, when the second surgical tool is placed in the vicinity of the patient's cornea, the computer processor is configured to drive display 24 to display an augmented second control-component tool 46, as shown in FIG. 2G. The augmented second control-component tool represents the second control-component tool. This augmented second control-component tool is typically a virtual representation of the second control-component tool.

Typically, in order to engage the second control-component tool with the second surgical tool (such that the movements of the second control-component tool control movement of the second surgical tool), the physician moves the second control-component tool such that the augmented second control-component tool is aligned with the image of second surgical tool 21 and/or augmented second surgical tool 44. Typically, the augmented second control-component tool is aligned with the image of second surgical tool 21 and/or augmented second surgical tool 44, such that (a) the tip of the augmented second control-component tool 46 overlays the image of second surgical tool 21 and/or augmented second surgical tool 44, and (b) the orientation of the augmented second control-component tool 46 is substantially similar to that of the image of second surgical tool 21 and/or augmented second surgical tool 44. Further typically, the computer processor is configured to position the augmented second control-component tool such that when the surgeon aligns the augmented second control-component tool with the image of second surgical tool 21 and/or augmented second surgical tool 44, the second control-component tool itself is disposed relatively centrally within the control-component workspace, and/or such that from this initial position within the control-component workspace the second control-component tool can be moved such as to move the second surgical tool to any location within the tool workspace, without the second control-component tool leaving the control-component workspace.

It is noted that it is typically not necessary for the augmented second control-component tool to be perfectly aligned with the image of second surgical tool 21 and/or augmented second surgical tool 44. Rather (as described with reference to the first surgical tool), in some applications, when the alignment is sufficiently close, any slight misalignment of position and/or orientation is maintained, such that the second surgical tool does not move at the moment of engagement, and thereafter follows the surgeon's movements with slight (and typically imperceptible) misalignment. For some applications, the computer processor drives the second robotic unit to adjust the position of the second surgical tool such as to complete the alignment of the augmented second control-component tool with the image of second surgical tool 21 and/or augmented second surgical tool 44.

Typically, once the augmented second control-component tool 46 is aligned with the image of second surgical tool 21 and/or augmented second surgical tool 44, the augmented second surgical tool 44 and/or the augmented second control-component tool 46 is removed from the image that is displayed by display 24. Typically, at this stage, the second control-component tool is engaged with the second surgical tool (such that the movements of the second control-component tool control movement of the second surgical tool). Typically, movements of the second control-component tool by the physician generate corresponding movements of the second surgical tool. Typically, rotation of the second control-component tool through angular rotations (roll, pitch, and/or yaw) generates corresponding rotations of the second surgical tool. Further typically, when the physician moves the second control-component tool translationally (along the X, Y, or Z directions) this generates corresponding translational motion of the second surgical tool. For some applications, the translational motion of the second surgical tool is scaled up or down relative to the translational motion of the second control-component tool.

Reference is now made to FIG. 21, which is a schematic illustration of display 24 showing augmented control-component tool 42 being moved away from augmented surgical tool 40 in order to disengage control-component tool 32 from surgical tool 21, in accordance with some applications of the present invention. For some applications, in order to disengage control-component tool 32 from surgical tool 21, the control-component tool is moved toward an edge of the control-component workspace and/or control-component tool is moved such that the surgical tool is moved toward the edge of the tool workspace. For some applications, the computer processor generates an indication of the disengagement before, during, and/or after the disengagement. For example, graphical elements 48 (e.g., stars, crosses, circles, highlights, or other graphical elements) may be displayed to indicate that the disengagement has happened, is happening, or is about to happen. Alternatively, a circle or an ellipse (not shown) is displayed around the iris (or at a different location), and the computer processor is configured to interpret a portion of the tool (such as the tip) exiting the circle as an indication that the operator wishes to disengage the control-component tool from the surgical tool. For such applications, the size of the circle or ellipse is typically selected such that it is visible within the field of view, but there would typically be no reason to move the portion of the tool outside of the circle for the purpose of the surgery.

Although the disengagement is shown in FIG. 2I with reference to the laterally-placed first surgical tool, typically generally similar steps are performed with reference to a superiorly-placed surgical tool.

As described hereinabove, the operator (e.g., physician 25A) typically assumes and relinquishes control of the surgical tool (via the control-component tool) multiple times during a procedure, especially when the procedure requires the use of multiple tools that are changed during surgery (as is typically the case with ophthalmic procedures, as described above). Typically, over the course of a procedure (a) the operator assumes control of the surgical tool, (b) the operator performs surgical actions with the surgical tool, (c) the operator relinquishes control of the surgical tool, (d) the robotic system or an operator (e.g., a nurse) removes the surgical tool from the robotic unit, (e), the robotic system or the operator places a new surgical tool on the robotic unit, and steps (a)-(e) are repeated. Typically, the each time the operator assumes control of a new tool, the steps described with reference to FIGS. 2A-2D or FIGS. 2E-2H are performed. Further typically, each time the operator relinquishes control of a tool, the steps described with reference to FIG. 2I are performed.

Typically, once the control-component tool becomes engaged with the surgical tool of the robotic unit, it does not disengage unless the physician performs a disengagement step (e.g., as described with reference to FIG. 21). However, it is noted that in some cases the control-component tool is disengaged from the surgical tool during a procedure, even without an input from the physician. For example, the control-component tool is disengaged from the surgical tool based on the robotic system detecting an imminent collision of tool with each other or with a portion of the patient's body. Typically, in the event that the control-component tool is disengaged from the surgical tool while the surgical tool is disposed within the tool workspace the physician re-engages the control-component tool with the surgical tool by performing the steps described hereinabove.

Reference is now made to FIG. 3A is a schematic illustration of the robotic system annotated with cuboids that indicate a control-component workspace 60 and a tool workspace 62 for illustrative purposes, in accordance with some applications of the present invention. As described hereinabove, typically, the control of the surgical tool by the operator has limitations. For example, control-component workspace 60 (i.e., the workspace in which the operator can move the control-component tool) is typically physically constrained by where it is comfortable or even possible for him/her to move the control-component tool. In addition, the tool workspace 62 (i.e., the workspace of the surgical tool) is typically physically constrained by the space within which it is possible for the robotic arm to move the surgical tool.

Control-component workspace 60 should be such that the control-component tool has sufficient freedom of movement such as to have the ability to control movement of the surgical tool within the surgical space. If the operator assumes control of the surgical tool (via the control-component tool) when the control-component tool is close to the edge of the control-component workspace, the movement of the control-component tool (and therefore that of the surgical tool) will be limited. Therefore, it is typically preferable for the operator to engage the control-component tool with the surgical tool when the control-component tool is positioned and oriented such that the operator has good freedom of movement of the control-component tool.

Tool workspace 62 should ideally cover the space within which the tool is expected to be manipulated for the purpose of the surgery (hereinafter “the surgical space”). If the operator assumes control of the surgical tool (via the control-component tool) when the surgical tool is at the edge of the tool workspace, the movement of the surgical tool will be limited. Therefore, it is typically preferable for the operator to engage the control-component tool with the surgical tool when the surgical tool is positioned and oriented such that it has good freedom of movement.

In other words, the operator should be able to freely move the surgical tool to all positions and orientation within the surgical space, without the control-component tool reaching the limits of the control-component workspace and without the surgical tool reaching the limits of the tool workspace. In accordance with some applications of the present invention, the control-component tool becomes engaged with the surgical tool, when the surgical tool and the control-component tool are toward the centers of the control-component workspace and the tool workspace, respectively.

For some applications, the control-component workspace has different dimensions from the tool workspace. For example, the tool workspace may be smaller than the control-component workspace, as shown. Typically, for such applications, movement of surgical tool 21 by robotic unit 20 are scaled down relative to movements of control-component tool 32.

Reference is now made to FIG. 3B, which is a schematic illustration of robotic system 10 annotated with several frames of references for illustrative purposes, in accordance with some applications of the present invention. Typically, there are several frames of reference that are operating within the robotic system and the computer processor transforms movements between the frames of reference. An example of this is now described with reference to FIG. 3B.

In the example shown in FIG. 3B, the display displays an image that corresponds to “superior” surgery—with the image that is shown to the physician being as if the surgeon is facing the patient's face, such that the patient's chin faces up and forehead faces down. Typically, in this configuration, when the physician moves the right control-component tool in the direction of his/her right hand within the control-component frame of reference F2, the right robotic unit moves surgical tool to the left (within right robotic unit frame of reference F5), and the surgical tool is moved to the right on display 24 (within display frame of reference F1). The display displays images captured by imaging system, with such images being acquired within the imaging system frame of reference F3. Similarly, movement of the left control-component tool causes the left robotic unit to move the left surgical tool (within left robotic unit frame of reference F4).

Typically, the control-component unit 26 is physically attached to the same body as display 24, such that there is a rigid and constant transformation from the control-component frame of reference F2 to the display frame of reference F1. The imaging system frame of reference F3 can be moved with respect to the patient. For example, the imaging system can be rotated such that it is as if the physician is viewing the eye with the superior and lateral (i.e., temporal) directions being reversed. Typically, whatever imaging system frame of reference is used, movements of the control-component tools within frame of reference F2 generates movement of the surgical tools in the same direction within display frame of reference F1.

To achieve proper transformations of movements between the frames of reference, the computer processor typically receives inputs that are indicative of the orientations of the various frames of reference relative to each other. For some applications, the computer processor analyzes images of the robotic units within images acquire by the imaging system in order to determine the orientations of the various frames of reference relative to each other. Alternatively or additionally, the computer processor receives an input from the operator indicating the orientation of surgery that she/he would like to be displayed on the display.

As described herein above, typically, the right control component controls movement of the surgical tool that is toward the right of the patient's head when viewing the patient from a superior position (and which would normally be controlled by the physician's right hand), and the left control component controls movement of the surgical tool that is toward the left of the patient's head when viewing the patient from a superior position (and which would normally be controlled by the physician's left hand). However, in some cases, the right control component controls movement of the surgical tool that is toward the left of the patient's head when viewing the patient from a superior position (and which would normally be controlled by the physician's left hand), and the left control component controls movement of the surgical tool that is toward the right of the patient's head when viewing the patient from a superior position (and which would normally be controlled by the physician's right hand). For example, in cases in which it would it is more intuitive to the physician and/or less physically cumbersome for the physician to use the left control-component tool to control the right surgical tool, and vice versa, rather than the other way round (e.g., based on the positions of the surgical tools and/or the handedness of the physician), the physician may switch which control component controls which tool. Alternatively or additionally, the computer processor or the physician may determine that it is easier for the surgical tools to perform their designated functions while staying within their ranges of motion by using the left control-component tool to control the right surgical tool and vice versa, and the computer processor may drive the robotic units to function accordingly (based upon the automatic detection by the computer processor or based upon an input from the physician). Typically, in such cases, the computer processor converts inputs from the physician regarding movements and actions that are provided within the right control-component tool frame of reference to corresponding movements and actions of the left surgical tool by the left robotic unit within the left robotic unit frame of reference, and/or vice versa.

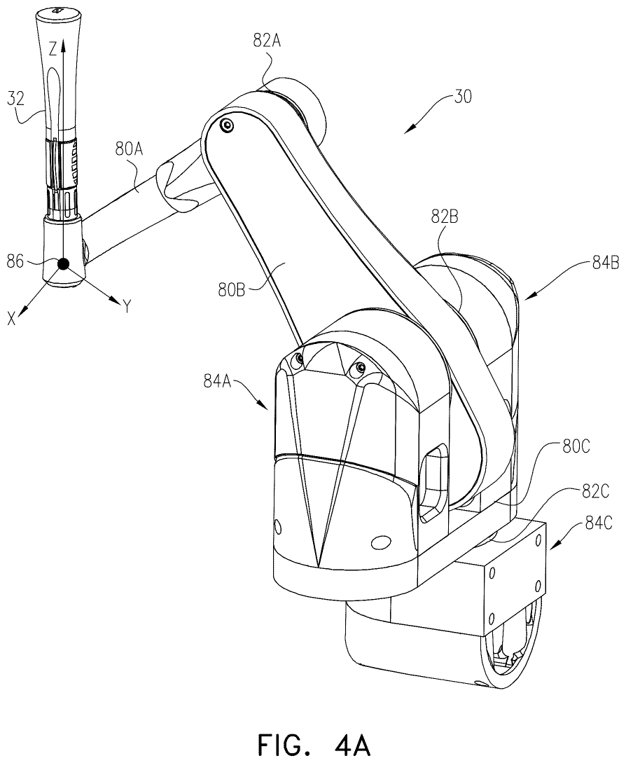

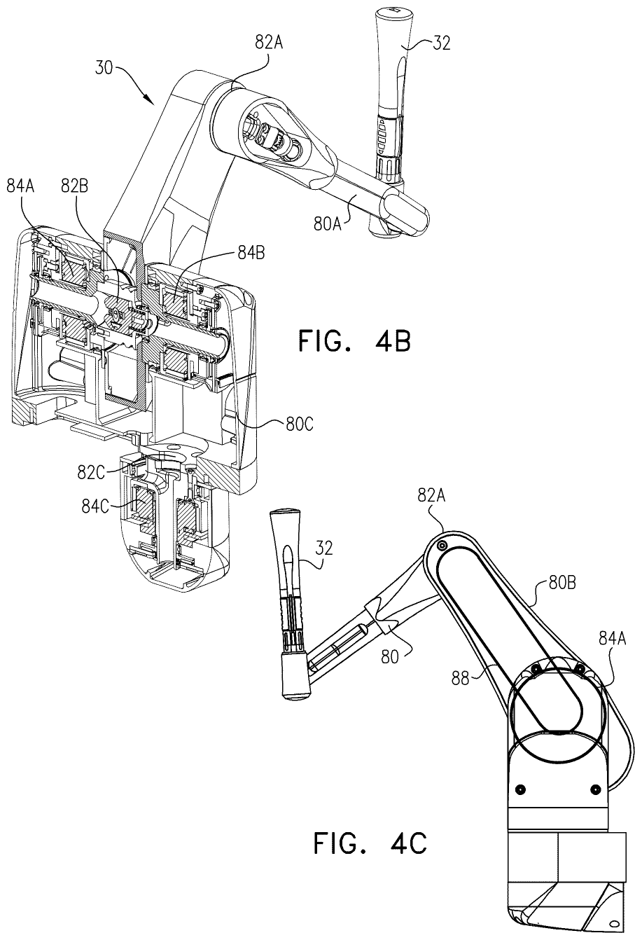

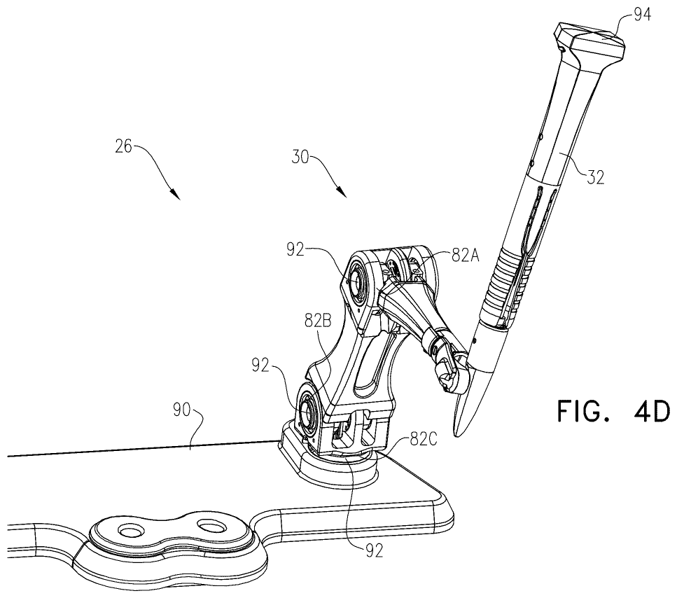

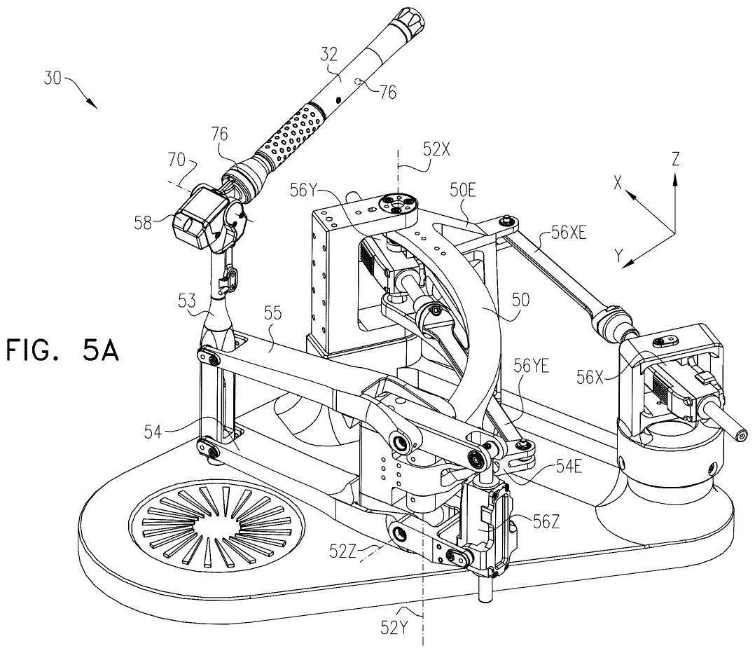

Reference is now made to FIGS. 4A, 4B, 4C, and 4D which are schematic illustrations of a control component 30 and control-component tool 32 of a control-component unit 26, in accordance with some applications of the present invention. As indicated in FIGS. 4A, 4B, and 4C, for some applications the control component is configured as a control-component arm that includes two or more links 80A, 80B, 80C that are connected via rotational arm joints 82A, 82B, 82C. For some applications, a respective motor 84A, 84B, 84C is configured to control movement of each of the rotational arm joints. For some applications, at least one of the motors (84A) applies torque to one of the rotational arm joints (82A) via a belt 88. Typically, a belt is used, such that the motor can be positioned closer to a base 90 of the control-component unit (base 90 being shown in FIG. 4D), in order to reduce the weight and inertia that the operator feels, relative to if the third motor were to be placed closer to rotational arm joint 82A. For some applications, a different configuration of motors is used within the control component.

For some applications, the motors are used to apply a force vector at a joint 86, at which the control-component tool is coupled to the control-component arm. Typically, the force vector is configured to counteract the force of gravity. In this manner, the operator is able to freely move the control-component tool without having to counteract the gravitational force that is generated by the weight of the control-component arm and/or by the control-component tool itself. For some applications, a force vector that is applied by the motors is calculated based upon the position of the control-component tool and the control-component arm, such that the force vector counterbalances the gravitational force that is generated by the weight of the control-component arm and the control-component tool in real time.

For some applications, the motors are used to apply a force vector such as to guide the operator from a docking position of the control-component tool to the position at which it becomes engaged with the surgical tool. In other words, referring back to FIGS. 2C-D, for some applications, the computer processor drives the motors to move the control-component tool such that the augmented control-component tool 42 is positioned such as to overlay augmented surgical tool 40. Typically, this reduces user fatigue that may be associated with performing this step manually and/or increases the speed with which this step is performed.

Referring to FIG. 4D, typically, in addition to the above-described motors, each of the control-component arms includes a respective rotary encoder 92 coupled to each one of the three rotational arm joints 82A, 82B, 82C. The rotary encoders are configured to detect movement of the respective rotational arm joints and to generate rotary-encoder data in response thereto. For some applications, the control-component arm additionally includes an inertial-measurement unit 94 that includes a three-axis accelerometer, a three-axis gyroscope, and/or a three-axis magnetometer. The rotary encoders and inertial-measurement unit are collectively referred to herein as “location sensors”. The inertial-measurement unit typically generates inertial-measurement-unit data relating to a three-dimensional orientation of the control-component arm, in response to the control-component arm being moved. For some applications, computer processor 28 receives the rotary-encoder data and the inertial-measurement-unit data. Typically, the computer processor determines the XYZ location of the tip of the control-component tool 32, based upon the rotary-encoder data, and determines the orientation of the control-component tool 32 (e.g., the 3 Euler angles of orientation, and/or another representation of orientation) based upon the inertial-measurement-unit data, or based upon a combination of the rotary-encoder data and the inertial-measurement-unit data. Thus, based upon the rotary-encoder data and/or the inertial-measurement-unit data, the computer processor is configured to determine the XYZ location and orientation of the control-component tool.

As described above, the control-component arm typically includes an inertial-measurement unit 94 that includes a three-axis accelerometer, a three-axis gyroscope, and/or a three-axis magnetometer. The accelerometers directly measure acceleration, gyroscopes directly measure angular velocity, and magnetometers measure magnetic fields. There are three of each type of sensor disposed in an orthogonal configuration with respect to each other, thus allowing 3D acceleration, angular velocity and magnetic field sensing.

The combination of the above measurements is used to infer the orientation of the inertial measurement unit. Specifically, algorithms generally use the earth's gravitational pull as a known acceleration, which is fused with the gyroscope measurements integrated over time to infer the inertial measurement unit's orientation. Without continuous correction of the orientation by the gravitational acceleration vector, the orientation output of the inertial measurement unit tends to drift. This is because angular information is derived from the gyroscopes through numerical integration. Any error in the angular velocity measurement accumulates over time, eventually resulting in a very poor estimate of the true orientation. The gravitational acceleration creates a “ground truth” that is used to remove the drift. If the gravitational vector did not change orientation, a change in angle derived from the gyroscopes can be ignored.

However, there is typically a possible rotation about the gravitational vector that cannot be sensed by the accelerometers. Since a rotation of the inertial measurement unit about the gravitational vector cannot be sensed by accelerometers, it can drift. One way to solve this drift is to fuse information from magnetometers, which provide a second “ground truth” vector that is linearly independent of the gravitational pull, i.e., magnetic North. However, this reading may be affected by other magnetic fields around the inertial measurement unit, which may cause errors in this reading.

In accordance with some applications of the present invention, a “ground truth” vector is derived that is linearly independent from the gravitational vector. Typically, the control-component tools are docked with respect to base in a given predetermined orientation, which is typically not vertical. For some applications, a sensor (such as a switch, a photo-reflector, etc.) identifies when the control-component tool is docked. When the control-component tool is docked the computer processor recalibrates the inertial measurement unit, based on two ground truth vectors—the gravitational vector and the known orientation of the control-component tool. Thus, each time the control-component tool is docked, the inertial measurement unit is recalibrated such that any drift is corrected.

For some applications, the computer processor performs the recalibration of the inertial measurement unit using the following algorithm. When the control-component tool is detected as being docked, the inertial measurement unit transmits the roll axis position that it is detecting to the computer processor. The roll axis is the axis that typically drifts due to lack of gravitational information. The roll axis position is cast onto the horizontal plane (normal to gravity), and compared to the known, true tilt angle; that is, the angle at which the control-component tool is known to lie within the horizontal plane. Whatever difference there is between the measured orientation and the true orientation is subtracted from the measured orientation. Effectively, the inertial measurement unit's measurement is corrected to fit the true orientation the control-component tool is known to be in, and the correction is maintained until the next time the control-component tool is docked, at which point the correction is repeated.

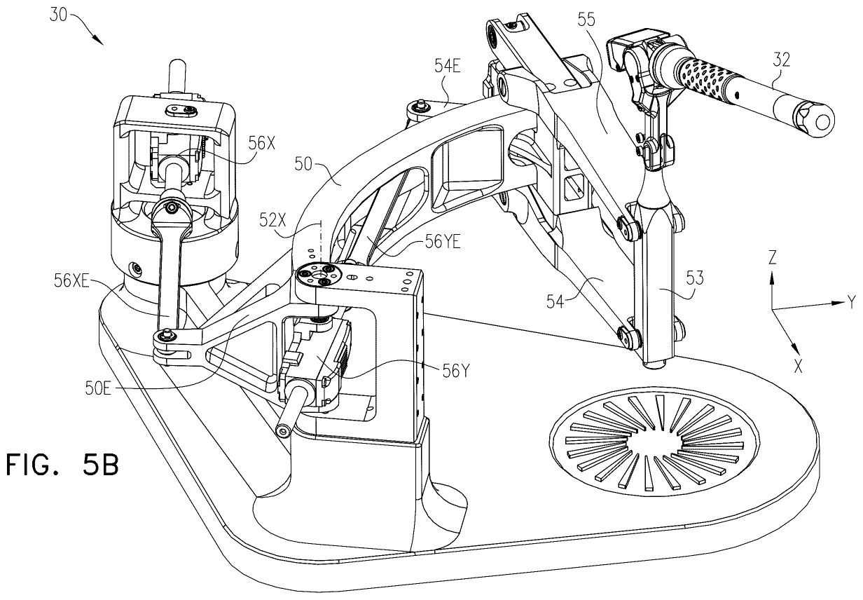

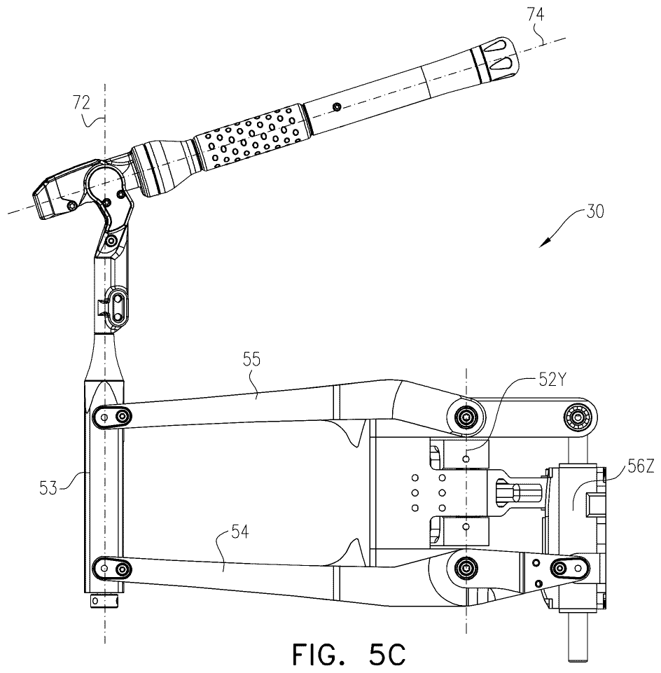

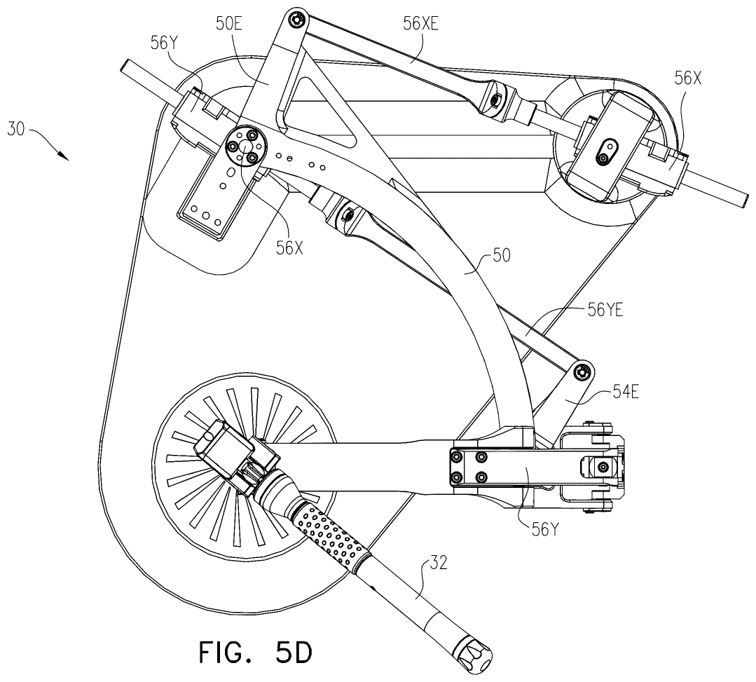

Reference is now made to FIGS. 5A, 5B, 5C, and 5D, which are schematic illustrations of control component 30 of a control-component unit, in accordance with some alternative applications of the present invention. FIGS. 5A and 5B show respective oblique views of the control component, FIG. 5C shows a side view, and FIG. 5D shows a top view. The functionality of control component 30 as shown in FIGS. 5A-5B is generally similar to that of control component 30 as shown in FIGS. 4A-4D except for the differences described hereinbelow.

Control-component 30 as shown in FIGS. 5A-5B typically includes a frame 50, which rotates around a first rotational axis 52X, and a link 54, which rotates around a second rotational axis 52Y and around a third rotational axis 52Z. Typically, the operator moving the control-component tool along X, Y, and Z linear directions causes links to rotate around respective rotational axes. For example, as the operator moves the control-component tool along the X linear direction, this causes frame 50 to rotate about rotational axis 52X, as the operator moves the control-component tool along the Y linear direction, this causes link 54 to rotate about rotational axis 52Y, and as the operator moves the control-component tool along the Z linear direction, this causes link 54 to rotate about rotational axis 52Z.

It is noted that the above description assumes that link 54 is disposed perpendicularly to frame 50. In practice, during much of the use of the control-component unit, link 54 is disposed at an angle to frame 50. In such configurations, movement of the control-component tool within the X-Y plane (even along the X linear direction or along the Y linear direction) will typically result in both frame 50 rotating about rotational axis 52X and link 54 rotating about rotational axis 52Y. For this reason, the use of the terms X, Y, and Z as used herein in relation to movements of portions of the control-component unit should not be interpreted as strictly corresponding to movement three linear axes that are perpendicular to each other. Rather, movement in the X and Y directions should be interpreted as relating to movement of frame 50 or link 54 within an X-Y plane (but not necessarily in directions that are perpendicular to each other) and movement in the Z direction should be interpreted as corresponding to movement of link 54 in a direction that is perpendicular to the X-Y plane. Thus, rotational axis 52X and motor 56X are associated with movement of frame 50 within the X-Y plane (regardless of whether the movement is in the X direction as indicated in the figures), rotational axis 52Y and motor 56Y are associated with movement of link within the X-Y plane (regardless of whether the movement is in the Y direction as indicated in the figures), and rotational axis 52Z and motor 56Z are associated with movement of link 54 perpendicularly to the X-Y plane.

Typically, as shown, the Y rotational axis 52Y is aligned with Z rotational axis 52Z along the Z direction. Further typically, both Y and Z linear motion are effected via link 54. It is noted that, for some applications, an additional supporting link 55 is disposed parallel to link 54 and rotates together with link 54. For some applications, link 54 and/or link 55 are made of two or more portions that are rigidly coupled to each other. For example, as shown in FIG. 5A, links 54 and 55 each includes a first portion disposed to the left of Z rotational axis 52Z, and a second portion disposed to the right of Z rotational axis 52Z. For some applications, a rotary encoder is disposed along each of the rotational axes 52X, 52Y, and 52Z (or a parallel rotational axis (e.g., the rotational axis of link 55). The rotary encoders detect rotation of respective links about the rotational axes, and generates signals in response thereto. The computer processor derives motion of the control-component tool along respective linear directions from the signal generated by the rotary encoders. For some applications, at least one additional rotary encoder is disposed along each of the rotational axes 52X, 52Y, and 52Z in order to provide the system with redundancy (e.g., such that in the event that one of the rotary encoders malfunctions, the other rotary encoder is used).

Typically, control-component tool 32 is moveable by the operator to undergo pitch, yaw, and roll angular rotations. The control-component tool typically undergoes pitch angular rotation by rotating about pitch rotational axis 70, and undergoes yaw angular rotation by a shaft 53 (upon which the control-component tool is mounted) rotating about its own axis 72 (which functions as the yaw rotational axis). Typically, the control-component tool undergoes roll angular rotation by rotating about its own axis 74 (which functions as the roll rotational axis). For some applications, an inertial-measurement unit 76 is housed within the control-component tool. Typically, the inertial measurement unit includes a three-axis accelerometer, a three-axis gyroscope, and/or a three-axis magnetometer. The inertial-measurement unit typically generates inertial-measurement-unit data relating to a three-dimensional orientation of the control-component tool. Alternatively or additionally, the control component includes one or more rotary encoders to detect the roll, pitch and/or yaw orientation of control-component tool 32. Typically, the rotary encoders are disposed along the axis about which the roll, pitch and yaw angular rotations occur, respectively. For some applications, the control component includes inertial-measurement unit 76 in addition to one or more rotary encoders to detect the roll, pitch and/or yaw of control-component tool 32, for redundancy (e.g., such that in the event that the inertial measurement unit malfunctions, the rotary encoders are used).

Typically, computer processor 28 receives the rotary-encoder data and the inertial-measurement-unit data. Typically, the computer processor determines the XYZ location of the tip of the control-component tool 32 based upon the rotary-encoder data, and determines the three-dimensional orientation of the tip of control-component tool 32 (e.g., the 3 Euler angles of orientation, and/or another representation of orientation) based upon the inertial-measurement-unit data, or based upon a combination of the rotary-encoder data and the inertial-measurement-unit data. Thus, based upon the combination of the rotary-encoder data and the inertial-measurement-unit data, the computer processor is configured to determine the XYZ location and three-dimensional orientation of the tip of the control-component tool.

Typically, a direct-drive motor 56X, 56Y, 56Z (i.e., a motor that does not impart motion via gear wheels), which is typically a linear motor (e.g., a linear voice coil motor), is associated with motion along the X, Y, and Z linear directions. For some applications, the computer processor is configured to drive the control-component unit to provide force feedback to the operator that is indicative of a location of the entry of the ophthalmic tool into the patient's eye within the incision. For some applications, the motors are configured to drive the tool to move linearly, in order to provide the aforementioned force feedback. For some applications, the computer processor is configured to apply forces that oppose the operator's attempted movements of control-component tool 32 that would result in violation of the remote center of motion. For example, in response to the operator moving the control-component tool through an angular yaw rotation that would cause a corresponding movement of the ophthalmic tool that would violate the remote center of motion, the computer processor may move the control-component tool linearly (through X, Y, and/or X linear motion) such that the remote center of motion of the ophthalmic tool is maintained. For some such applications, the forces are applied by driving the control-component tool to move in the X, Y, and Z linear directions, via motors 56X, 56Y, 56Z.

Typically, robotic system 10 is used in procedures that require delicate and precise movements of the surgical tools, e.g., ophthalmic procedures, as described hereinabove. Therefore, control component 30 is typically configured such that movement of control-component tool is performed by the operator without there being substantial counterforces to the movement (other than counterforces that are deliberately applied via motors 56X, 56Y, 56Z). For some applications, the control-component tool includes a counterweight 58, such that the weight of the control-component tool is relatively evenly balanced about pitch rotational axis 70. For some applications, the control-component tool is not entirely balanced about pitch rotational axis 70, in order to give the physician a feeling of the tool's weight (like a real surgical tool), and/or also to reduce the overall mass of the control-component tool. For some applications, link 54 extends across both sides of the Z rotational axis 52Z, with the control-component tool and additional components being disposed on link 54 (and/or parallel link 55) on a first side of rotational axis 52Z. For some applications, motor 56Z, which is disposed along the Z linear directions is disposed on link 54 on the other side of rotational axis 52Z, such as to balance the weight of the control-component tool and additional components that are disposed on the first side. For some such application, the control-component unit does not include an additional counterweight for this purpose. Alternatively, the control-component unit include a counterweight for this purpose, in addition to motor 56Z.

For some applications, frame 50 (which functions as the link through which X direction linear motion is effected) comprises two curved arms and motor 56Y (and, optionally, an extension 56YE thereof) passes between the two curved arms along a straight line. For some applications, an end of frame 50 which is adjacent to Z rotational axis 52Z is aligned with Z rotational axis 52Z (as shown in FIG. 5A), such that frame 50 does not exert any torque about Z rotational axis 52Z. Thus, frame 50 does not need to be counterbalanced about Z rotational axis 52Z. For some applications, even as frame 50 moves (due to motion in the X direction), the frame remains aligned with Z rotational axis 52Z, such that no compensatory motion is necessary in order to balance the motion of the frame.

It is noted that in accordance with the above description, the control-component unit typically is balanced within all six degrees of freedom (the three axial translations and three angular rotations). For some applications, the control-component unit utilizes counterweights to provide balance in two degrees of freedom: Z direction axial motion and pitch angular motion. In the embodiment shown in FIGS. 5A-5D, motor 56Z functions as the counterweight in the Z direction axial motion degree of freedom. The remaining four degrees of freedom (i.e., X and Y axial motion, and roll and yaw angular motions) typically do not require counterweights for balancing, since the control-component unit is designed such that the control-component tool and/or other elements of the control-component unit are self-balancing within these degrees of freedom. Since the control-component unit is designed to be balanced within all six degrees of freedom (e.g., by self-balancing within four degrees of freedom and balance being provided by the counterweights within the two remaining degrees of freedom), the control-component tool tends to maintain its position and orientation, in the absence of any forces acting upon the control-component tool. Thus, typically if the operator temporarily lets go of the control-component tool (without exerting force on the control-component tool as she/he lets go of the tool), the control-component tool maintains its position and orientation until the operator resumes control of the control-component tool. Further typically, the control-component tool is able to provide force feedback to the operator at relatively low levels of force, since the control-component tool provides relatively low inertial forces. I.e., the motors that are configured to provide force feedback to the operator by driving the control-component tool to move are configured to do so substantially without being required to overcome inertial forces.

For some applications, as shown in FIGS. 5A-5B, motor 56Y is disposed within the X-Y plane such that its center of mass is substantially aligned with X rotational axis 52X both when motor 56Y is extended and when motor 56Y retracted. Typically, this prevents movement of motor 56Y from exerting any torque in the Z direction on link 54 as motor 56Y extends and contracts. It is noted that as the motor extends and contracts, its center of mass moves slightly. Typically, the motor is positioned such that in at least one position within its fully extended and fully contracted states, the motor's center of mass is aligned with X rotational axis 52X. Further typically, the motor's center of mass is aligned with X rotational axis 52X, when the motor is at its central position with respect to its fully extended and fully retracted states. For some applications, both when the motor is fully extended and fully retracted, its center of mass is within 10 mm, e.g., within 5 mm of X rotational axis 52X. It is further noted that motor 56Y is typically coupled to frame 50, such that motor 56Y is configured to rotate together with frame 50. By being configured in this manner, the motor does not apply any torque to frame 50 even as frame 50 rotates.

For some applications, frame 50 includes an angled extension 50E to which motor 56X (and, optionally, an extension 56XE thereof) is coupled. Motor 56X rotates frame 50 about axis 52X by the motor (or the extension thereof) pushing or pulling angled extension 50E. Typically, by the control-component unit incorporating angled extension 50E, the dimensions of the control-component unit (and the overall footprint of the control component) are reduced relative to if motor 56X (or extension 56XE thereof) were to be coupled to non-angled continuation of frame 50 on an opposite side of axis 52X from the main portion of frame 50. For some applications (not shown), motor 56X rotates frame 50 about axis 52X by the motor (or the extension thereof) by pushing or pulling a non-angled extension that is disposed within the footprint of the frame.

Similarly, for some applications, link 54 includes an angled extension 54E to which motor 56Y (and, optionally, an extension 56YE thereof) is coupled. Motor 56Y rotates link 54 about axis 52Y by the motor (or the extension thereof) pushing or pulling angled extension 54E. Typically, by the control-component unit incorporating angled extension 54E, the dimensions of the control-component unit (and the overall footprint of the control component) are reduced relative to if motor 56Y (or extension 56YE thereof) were to be coupled to non-angled continuation of link 54 on an opposite side of axis 52Y from the main portion of link 54. For some applications (not shown), motor 56Y rotates frame 50 about axis 52Y by the motor (or the extension thereof) by pushing or pulling link 54 at location that is offset from the Y rotational axis 52Y.

For some applications, longitudinal axis 72 of shaft 53 (which functions as the yaw rotational axis) is aligned with the ends of links 54 and 55.

As described with reference to FIGS. 4A-4D, typically, the control-component tools are docked with respect to base in a given predetermined orientation, which is typically not vertical. For some applications, a sensor (such as a switch, a photo-reflector, etc.) identifies when the control-component tool is docked. When the control-component tool is docked the computer processor recalibrates the inertial measurement unit, based on two ground truth vectors—the gravitational vector and the known orientation of the control-component tool. Thus, each time the control-component tool is docked, the inertial measurement unit is recalibrated such that any drift is corrected.

For some applications, the computer processor performs the recalibration of the inertial measurement unit using the algorithm described hereinabove with reference to FIGS. 4A-4D. When the control-component tool is detected as being docked, the inertial measurement unit transmits the roll axis position that it is detecting to the computer processor. The roll axis is the axis that typically drifts due to lack of gravitational information. The roll axis position is cast onto the horizontal plane (normal to gravity), and compared to the known, true tilt angle; that is, the angle at which the control-component tool is known to lie within the horizontal plane. Whatever difference there is between the measured orientation and the true orientation is subtracted from the measured orientation. Effectively, the inertial measurement unit's measurement is corrected to fit the true orientation the control-component tool is known to be in, and the correction is maintained until the next time the control-component tool is docked, at which point the correction is repeated.

Although some applications of the present invention are described with reference to cataract surgery, the scope of the present application includes applying the apparatus and methods described herein to other medical procedures, mutatis mutandis. In particular, the apparatus and methods described herein to other medical procedures may be applied to other microsurgical procedures, such as general surgery, orthopedic surgery, gynecological surgery, otolaryngology, neurosurgery, oral and maxillofacial surgery, plastic surgery, podiatric surgery, vascular surgery, and/or pediatric surgery that is performed using microsurgical techniques. For some such applications, the imaging system includes one or more microscopic imaging units.

It is noted that the scope of the present application includes applying the apparatus and methods described herein to intraocular procedures, other than cataract surgery, mutatis mutandis. Such procedures may include collagen crosslinking, endothelial keratoplasty (e.g., DSEK, DMEK, and/or PDEK), DSO (descemet stripping without transplantation), laser assisted keratoplasty, keratoplasty, LASIK/PRK, SMILE, pterygium, ocular surface cancer treatment, secondary IOL placement (sutured, transconjunctival, etc.), iris repair, IOL reposition, IOL exchange, superficial keratectomy, Minimally Invasive Glaucoma Surgery (MIGS), limbal stem cell transplantation, astigmatic keratotomy, Limbal Relaxing Incisions (LRI), amniotic membrane transplantation (AMT), glaucoma surgery (e.g., trabs, tubes, minimally invasive glaucoma surgery), automated lamellar keratoplasty (ALK), anterior vitrectomy, and/or pars plana anterior vitrectomy.

Applications of the invention described herein can take the form of a computer program product accessible from a computer-usable or computer-readable medium (e.g., a non-transitory computer-readable medium) providing program code for use by or in connection with a computer or any instruction execution system, such as computer processor 28. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W), DVD, and a USB drive.

A data processing system suitable for storing and/or executing program code will include at least one processor (e.g., computer processor 28) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the invention.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present invention may be written in any combination of one or more programming languages, including an object-oriented programming language such as Java, Smalltalk, C++ or the like and conventional procedural programming languages, such as the C programming language or similar programming languages.

It will be understood that the algorithms described herein, can be implemented by computer program instructions. These computer program instructions may be provided to a processor of a general-purpose computer, special purpose computer, or other programmable data processing apparatus to produce a machine, such that the instructions, which execute via the processor of the computer (e.g., computer processor 28) or other programmable data processing apparatus, create means for implementing the functions/acts specified in the algorithms described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the algorithms. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the algorithms described in the present application.

Computer processor 28 is typically a hardware device programmed with computer program instructions to produce a special purpose computer. For example, when programmed to perform the algorithms described with reference to the Figures, computer processor 28 typically acts as a special purpose robotic-system computer processor. Typically, the operations described herein that are performed by computer processor 28 transform the physical state of a memory, which is a real physical article, to have a different magnetic polarity, electrical charge, or the like depending on the technology of the memory that is used. For some applications, operations that are described as being performed by a computer processor are performed by a plurality of computer processors in combination with each other.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. Apparatus for performing a procedure on a portion of a body of a patient using a surgical tool that has a tip, an imaging system and a display, the apparatus comprising:
a robotic unit configured to move the surgical tool;
a control-component unit that comprises one or more location sensors and a control-component tool that is configured to be moved by an operator and that defines a tip; and
at least one computer processor configured:
to drive the display to show an image that includes a representation of the surgical tool;
in response to the control-component tool being at least partially aligned with the representation of the surgical tool within the image upon the display, to engage the control-component tool with the surgical tool, and
when the control-component tool is engaged with the surgical tool:
to determine movement of the location and orientation of the tip of the control-component tool based upon data received from the one or more location sensors; and
to move the tip of the surgical tool within the patient's eye in a manner that corresponds with the movement of the location and orientation of the tip of the control-component tool.

2. The apparatus according to claim 1, wherein the apparatus is configured for performing an ophthalmic procedure on an eye of a patient using one or more ophthalmic tools that have tips and wherein the robotic unit is configured to move the one or more ophthalmic tools within the patient's eye.

3. The apparatus according to claim 1, wherein the computer processor is configured to drive the display to display an augmented control-component tool overlaid upon the control-component tool upon the display, and wherein the computer processor is configured to engage the control-component tool with the surgical tool in response to the augmented control-component tool being at least partially aligned with the representation of the surgical tool within the image upon the display.

4. The apparatus according to claim 1, wherein the computer processor is configured to automatically move the control-component tool to become at least partially aligned with the representation of the surgical tool within the image upon the display, such that the control-component tool becomes engaged with the surgical tool.

5. The apparatus according to claim 1, wherein the robotic unit is capable of moving the surgical tool within a tool workspace, and wherein the computer processor is configured to automatically drive the robotic unit to move the surgical tool to an initial position at which the surgical tool is within a given portion of the tool workspace.

6. The apparatus according to claim 1, wherein:
the robotic unit is configured to move the surgical tool within a tool frame of reference;
the control-component tool is moveable within a control-component frame of reference; and
the computer processor is configured to guide the operator to move the control-component tool to become engaged with the surgical tool when the control-component tool is disposed at an orientation within the control component frame of reference that is substantially similar to an orientation of the surgical tool within the tool frame of reference.

7. The apparatus according to claim 1, wherein the computer processor is configured to engage the control-component tool with the surgical tool, without requiring any inputs via any operator-controlled interfaces other than movement of the control-component tool.

8. The apparatus according to claim 1, wherein the computer processor is configured to drive the display to show an image that includes a representation of the surgical tool by driving the display an image of the surgical tool.

9. The apparatus according to claim 8, wherein the computer processor is configured to drive the display to show an image that includes a representation of the surgical tool by driving the display an image of the surgical tool and the portion of the patient's body.

10. The apparatus according to claim 8, wherein the computer processor is configured to drive the display to show an image that includes a representation of the surgical tool by driving the display to display an augmented surgical tool overlaid upon the surgical tool upon the display.

11. The apparatus according to claim 1, wherein:
the surgical tool includes left and right surgical tools;
the robotic unit comprises left and right robotic units configured to move the left and right surgical tools respectively;
the control-component unit comprises left and right control-component tools that are configured to be moved by the operator and that define tips;
the left control-component tool is engageable with both of the left and right surgical tools; and
the right control-component tool is engageable with both of the left and right surgical tools.

12. The apparatus according to claim 11, wherein the computer processor is configured to engage the left control-component tool with the right surgical tool and the right control-component tool with the left surgical tool in response to an input from the operator.

13. The apparatus according to claim 11, wherein the computer processor is configured to engage the left control-component tool with the right surgical tool and the right control-component tool with the left surgical tool automatically based on positions of the left and right robotic units relative to the portion of the patient's body.

14. The apparatus according to claim 1, wherein the robotic unit is capable of moving the surgical tool within a tool workspace, and wherein the computer processor is configured to disengage the control-component tool from the surgical tool in response to the surgical tool being moved toward an edge of the tool workspace.

15. The apparatus according to claim 14, wherein the computer processor is configured to generate a graphic on the display indicating that the control-component tool is being disengaged from the surgical tool.

16. The apparatus according to claim 1, wherein the control-component tool is moveable within a control-component workspace, and wherein the computer processor is configured to guide the operator to move the control-component tool to become engaged with the surgical tool when the control-component tool is disposed within a given portion of the control-component workspace.

17. The apparatus according to claim 16, wherein the computer processor is configured to guide the operator to move the control-component tool to become engaged with the surgical tool when the control-component tool is disposed relatively centrally within the control-component workspace.

18. The apparatus according to claim 16, wherein the robotic unit is capable of moving the surgical tool within a tool workspace, and wherein the computer processor is configured to guide the operator to move the control-component tool to become engaged with the surgical tool when the control-component tool is disposed such that the control-component tool can be moved so as to move the surgical tool to any location within the tool workspace without the control-component tool leaving the control-component workspace.

19. Apparatus for performing a procedure on a portion of a body of a patient using a surgical tool that has a tip, an imaging system and a display, the apparatus comprising:
a robotic unit configured to move the surgical tool within a tool frame of reference;
a control-component unit that comprises one or more location sensors and a control-component tool that is configured to be moved by an operator and that defines a tip, and that is moveable within a control-component frame of reference; and
at least one computer processor configured:
to guide the operator to move the control-component tool to become engaged with the surgical tool when the control-component tool is disposed at an orientation within the control-component frame of reference that is substantially similar to an orientation of the surgical tool within the tool frame of reference, and
when the control-component tool is engaged with the surgical tool:
to determine movement of the location and orientation of the tip of the control-component tool based upon data received from the one or more location sensors; and
to move the tip of the surgical tool within the patient's eye in a manner that corresponds with the movement of the location and orientation of the tip of the control-component tool.

20. The apparatus according to claim 19, wherein the computer processor is configured to engage the control-component tool with the surgical tool, without requiring any inputs via any operator-controlled interfaces other than movement of the control-component tool.

21. The apparatus according to claim 19, wherein the robotic unit is capable of moving the surgical tool within a tool workspace, and wherein the computer processor is configured to disengage the control-component tool from the surgical tool in response to the surgical tool being moved toward an edge of the tool workspace.

22. The apparatus according to claim 19, wherein the computer processor is configured to drive the display to show an image that includes a representation of the of the surgical tool.

23. The apparatus according to claim 22, wherein the computer processor is configured, in response to the control-component tool being at least partially aligned with the representation of the surgical tool within the image upon the display, to engage the control-component tool with the surgical tool.

24. The apparatus according to claim 19, wherein the control-component tool is moveable within a control-component workspace, and wherein the computer processor is configured to guide the operator to move the control-component tool to become engaged with the surgical tool when the control-component tool is disposed within a given portion of the control-component workspace.

25. The apparatus according to claim 24, wherein the computer processor is configured to guide the operator to move the control-component tool to become engaged with the surgical tool when the control-component tool is disposed relatively centrally within the control-component workspace.

26. The apparatus according to claim 24, wherein the robotic unit is capable of moving the surgical tool within a tool workspace, and wherein the computer processor is configured to guide the operator to move the control-component tool to become engaged with the surgical tool when the control-component tool is disposed such that the control-component tool can be moved such as to move the surgical tool to any location within the tool workspace without the control-component tool leaving the control-component workspace.
